(19) United States
(12) Patent Application Publication
(10) Pub. No.: US 2010/0015008 A1
(43) Pub. Date: Jan. 21, 2010

(54) MICROFLUIDIC DEVICE FOR ANALYZING THE STATUS OF A PARTICLE

(75) Inventors: Wee Liat Ong, Singapore (SG); Levent Yobas, Singapore (SG); Kim Cheong Tang, Singapore (SG); Ajay Agarwal, Singapore (SG); Nagarajan Ranganathan, Singapore (SG)

Correspondence Address: CONNOLLY BOVE LODGE & HUTZ LLP, 1875 EYE STREET, N.W., SUITE 1100, WASHINGTON, DC, 20006, US

(73) Assignee: AGENCY FOR SCIENCE, TECHNOLOGY AND RESEARCH, Centros (SG)

(21) Appl. No.: 12/294,205

(22) PCT Filed: Mar. 23, 2007
(86) PCT No.: PCT/SG07/00080

§ 371 Date: Aug. 12, 2009

(30) Foreign Application Priority Data
Mar. 23, 2006 (SG) PCT/SG2006/000071

(51) Int. Cl. G01N 27/00; G01N 33/00; B29B 15/10

(52) U.S. Cl. 422/82.01; 422/68.1; 264/340

(57) ABSTRACT

The present invention provides a device for analyzing the status of a biological entity. The device comprises a base substrate having a recess defined therein by two opposing lateral walls and a base wall, a filler member having at least a portion thereof occupying the recess, and a channel defined in the portion of the filler member occupying the recess, wherein the channel comprises a first aperture and a second aperture, the first aperture being arranged on a first lateral wall of the filler member, and the second aperture being arranged on a second lateral wall of the filler member, said first lateral wall of the filler member being arranged in opposing relationship with the second lateral wall of the filler member, and at least a portion of the first and the second lateral walls of the filler member being at least substantially perpendicular to the opposing lateral walls defining the recess.

### CROSS-REFERENCE TO RELATED APPLICATIONS

The present application makes reference to and claims the benefit of priority of an application for a “Device for analyzing the status of a particle”, filed Mar. 23, 2006 as international patent application PCT/SG2006/000071, the contents of which is incorporated herein by reference in its entirety for all purposes.

### FIELD OF THE INVENTION

The present invention relates generally to a microfluidic device for analyzing the status of a particle, its formation and its use, and particularly to a sensor that can be used for the detection of a biological entity such as a living cell.

### BACKGROUND OF THE INVENTION

For many years, scientific studies on transport activity in cell membranes have required the use of the patch clamp devices. This technique directly monitors the ionic current through membrane proteins while holding the membrane potential at a bias voltage. The current signal contains information on behaviour of ion channels which is crucial particularly in nerve and muscle physiology. Abnormalities with ion channels lead to diseases like cystic fibrosis, epilepsy, myotonia, and osteopetrosis. Potential cures for such diseases are typically screened against the target ion channels using indirect assays since they offer higher throughput than the otherwise superior patch clamping. Therefore, other technologies such as radioligand binding, fluorescence measurement of ions or membrane potential or atomic absorption measurements of ions have been established as industrial standard methods in drug development for ion channels as targets. Measurements made using these patch clamp devices have nevertheless provided a direct and accurate way of monitoring a cell's behaviour, in particular due to the method's high time resolution, high sensitivity, and the available options of configurations for measurement. For this reason, patch clamp devices have become indispensable tools and been extensively used in many areas, especially in the screening of pharmaceutical compounds, in which the effect of a drug on a cell can be determined relatively accurately.

In a patch clamp test, an extremely fine pipette (also known as a micropipette) is held tightly against the cell membrane to record its electrical activity. However, limitations in instrumentation present several problems which hinder the effective use of patch clamp devices. For example, in a typical patch clamp test procedure, a human operator needs to carry out precision physical manoeuvres involving a small glass micropipette using micromanipulators while visually monitoring the pipette tip and biological cell under an optical microscope. The procedure involved in manipulating the micropipette and carrying out measurements from the micropipette is a skill-laden and delicate procedure that creates a bottleneck in the screening process, especially if hundreds of drugs are to be tested, thereby causing low throughput.

Another major problem encountered in implementing conventional patch clamp devices is the difficulty in obtaining stable seals between the glass micropipette and the sample. Ideally, a stable seal completely isolates micropipette fluid (inside the micropipette) from bath fluid (outside the micropipette) with minimal ion leakage at the interface between the sample and the patch aperture. Seals which are able to achieve high electrical resistance, in particular in the order of giga-ohms, can accurately record pico-ampere currents (due to the movement of ions) through the patch sample without being affected by noise signals in the background.

In order to overcome shortcomings of conventional micropipette patch clamp devices, horizontally orientated planar patch-clamps have been proposed. Planar patch-clamp chips provide an insulated partition, mostly a thin diaphragm, through which a patch aperture is defined. The partition separates bath fluid on one side from pipette fluids on the other side, while a gentle suction applied through the patch aperture attracts and eventually immobilises the particle to be tested.

However, the planar patch clamps have several drawbacks. For example, the packaging of planar patch clamp devices requires multiple-layer alignment and bonding in order to isolate fluids located both above and bottom of the device substrate. The fabrication of such devices imposes difficulties, in particularly when an array is desired. Additionally, fabrication turnaround may be longer as the entire chip substrate typically needs to be etched away. Although the patch aperture is less than 2 μm in diameter, usually a chip area of 1×1 mm² has to be etched to accommodate a corresponding diaphragm. This requirement leads to a lower density array.

An alternative patch clamp device has been suggested in response to the difficulties encountered in planar patch clamps. This alternative device comprises arranging the patch channel laterally within a vertical wall, and the patch aperture therefore positioned within the vertical plane of the wall. By implementing a lateral patch aperture, fluidic structures can be arranged laterally, thereby avoiding the difficulties associated with a vertically built-up structure in which fluid partitioning is problematic, especially if the device is scaled up to include arrays of test chambers.

However, the fabrication of lateral patch apertures presents several problems. For example, there are difficulties in achieving a patch aperture with circular geometry because micromachining techniques applicable to planar patch apertures are based on planar lithography. Some attempts have been made to address this problem.

Seo et al. (App. Phys. Lett. (2004) 84, 11, 1973-1975) describe an integrated multiple patch clamp array chip. The chip utilises lateral cell trapping junctions having patch channels arranged within a wall which separates the cell reservoir from a suction chamber from which sample fluid is drawn to provide suction force which immobilises the cell onto the patch aperture. However, the chip was fabricated from polydimethylsiloxane micro-molding and produced only semi-circular apertures. One shortcoming of patch clamping a cell using a patch clamp device that does not have a round patch aperture is that they do not achieve seal resistances in the range of giga-ohms. Accordingly, measurements taken from the device required the use of leakage subtraction software which may not model the actual test environment accurately.

US patent application 2003/0180965 discloses a microfluidic device with a micropipette fabricated using semiconductor manufacturing techniques. The micropipette of the device, which is suitable for usage as a patch-clamp setup, is created using complex cycles of etching and deposition. A final removal of a created layer from underneath another layer generates a micropipette-like structure.

Tjerkstra et al. (Tenth Annual International Workshop on Micro Electro Mechanical Systems, 1997, MEMS '97, 26-30 Jan. 1997, Proceedings, IEEE, 147-152, DOI: 10.1109/MEMSYS.1997.581790) discloses the use of a combination of wet and dry anisotropic and isotropic silicon etching processes, followed by low pressure chemical vapour deposition (LPCVD) sealing or glass wafer bonding. Channels in silicon wafer are realised by first forming a straight recess (anisotropic silicon etch) followed by circular recess (isotropic etch). The surface of the wafer is subsequently covered with silicon nitride. A sealing layer is deposited onto the silicon nitride layer. The silicon nitride in the trench is subsequently etched away using reactive ion etching (RIE), leaving behind a channel.

Accordingly, it is an object of the present invention to provide a device which overcomes some of the drawbacks of the prior art devices, in particular to provide a device which is more suitable for conventional patch clamp applications.

### SUMMARY OF THE INVENTION

According to a first aspect of the present invention, a microfluidic device is provided. The microfluidic device includes a base substrate having defined therein a recess. The recess is defined in the base substrate by two or at least two opposing lateral walls and a base wall. The device also includes a filler member having at least a portion thereof occupying the recess. The device further includes a channel. The channel includes a first aperture and a second aperture, for instance a first and a second opening. The first aperture is arranged on a first lateral wall of the filler member. The second aperture is arranged on a second lateral wall of the filler member. Furthermore the first lateral wall of the filler member is arranged in opposing relationship with the second lateral wall of the filler member. At least a portion of the first and the second lateral walls of the filler member is at least substantially perpendicular to the opposing lateral walls defining the recess.

In a second aspect the invention provides a microfluidic device. The microfluidic device includes a base substrate. The base substrate includes a recess. The recess includes two opposing lateral walls and a base wall. The microfluidic device further includes a filler member. A portion of the filler member is included in the recess of the base substrate. The microfluidic device further includes a channel defined in the portion of the filler member that is included in the recess. The channel includes a first aperture and a second aperture. The first aperture is arranged on a first lateral wall of the filler member. The second aperture is arranged on a second lateral wall of the filler member. Furthermore the first lateral wall of the filler member is arranged in opposing relationship with the second lateral wall of the filler member. At least a portion of the first and the second lateral walls of the filler member is at least substantially perpendicular to the opposing lateral walls defining the recess. The channel is defined by a circumferential wall. The circumferential wall of the channel has a portion that is contiguous to the first aperture. This portion is conical along its length in that the size of the circumferential wall in terms of its width decreases toward the first aperture. According to some embodiments this portion defines a tip.

In a third aspect the invention provides a further microfluidic device. The microfluidic device includes a first and a second fluid chamber. The first fluid chamber is for containing a particle to be tested. The second fluid chamber is fluidly separated from the first fluid chamber by means of a partitioning element. The partition element corresponds to a device as defined above. It includes a base substrate having a recess defined therein. It also includes a filler member having a portion thereof occupying the recess. The partition element further includes a channel defined in the portion of the filler member occupying the recess. The channel includes a first aperture and a second aperture. The first aperture is arranged on a first lateral wall of the filler member. The second aperture is arranged on a second lateral wall of the filler member. The first lateral wall of the filler member is arranged in opposing relationship with the second lateral wall of the filler member. At least a portion of the first lateral wall and the second lateral wall of the filler member are at least substantially perpendicular to the opposing lateral walls defining the recess.

According to a fourth aspect the invention provides a further microfluidic device. The microfluidic device includes a first and a second fluid chamber. The first fluid chamber is for containing a particle to be tested. The second fluid chamber is fluidly separated from the first fluid chamber by means of a partitioning element. The partition element corresponds to a device as defined above. It includes a base substrate having a recess defined therein. The partition element further includes a filler member having a portion thereof occupying the recess. It also includes a channel defined in the portion of the filler member occupying the recess. The channel includes a first aperture and a second aperture. The first aperture is arranged on a first lateral wall of the filler member. The second aperture is arranged on a second lateral wall of the filler member. The first lateral wall of the filler member is arranged in opposing relationship with the second lateral wall of the filler member. The channel is defined by a circumferential wall. The circumferential wall of the channel has a portion that is contiguous to the first aperture. This portion is conical along its length in that the size of the circumferential wall in terms of its width decreases toward the first aperture. According to some embodiments this portion defines a tip.

In a fifth aspect the invention relates to a method of forming a device of the invention according to one of the first to the fourth aspects. The method includes providing a base substrate for forming the device. A recess is formed on a surface of the substrate. Furthermore the recess is partially filled with a filling material. The filling material is subjected to a condition that causes it to deform. Thereby a channel is formed within the filler member.

This method allows in particular embodiments of forming a channel in an open-ended recess that stretches up to at least one side of the base substrate. In such embodiments the formed channel can have a circumferential wall with a portion adjacent to the open-ended side of the recess. This portion is conical along its length in that the size of the circumferential wall in terms of its width decreases toward the open-ended side of the recess.

According to a sixth aspect of the invention, there is provided a further method of forming a device of the invention according to one of the first to the fourth aspects. The method includes providing a base substrate. The method also includes forming a first recess on a surface of the base substrate. The method further includes forming a second recess on a surface of the base substrate. This surface of the base substrate differs from the surface on which the first recess is formed. The method also includes filling the first recess with the filling material. Further the method includes subjecting the filling material to a condition that causes it to deform. Thereby a channel is formed in the portion of the filling material that occupies the first recess.

This method allows in particular embodiments of forming a channel in a recess that is open-ended in that the recess stretches up to at least one side of the base substrate. In such embodiments the formed channel can have a circumferential wall with a portion adjacent to the open-ended side of the recess. This portion is conical along its length in that the size of the circumferential wall in terms of its width decreases toward the open-ended side of the recess.

In a seventh aspect the invention relates to a method of forming a device of the invention according to one of the first to the fourth aspects. The method includes providing a base substrate. Further the method includes forming a recess on a surface of the base substrate. The recess is formed in such a way that it is open-ended in that it stretches up into at least one side of the base substrate. The method further includes covering the base substrate with a filling material. The method also includes subjecting the filling material to a condition that causes it to deform. Thereby a channel is formed in the portion of the filling material that occupies the second recess.

This method allows in particular embodiments of forming a channel in a recess that is open-ended in that the recess stretches up to at least one side of the base substrate. In such embodiments the formed channel can have a circumferential wall with a portion adjacent to the open-ended side of the recess. This portion is conical along its length in that the size of the circumferential wall in terms of its width decreases toward the open-ended side of the recess.

According to an eighth aspect of the invention, there is provided a method of analyzing the status of a biological entity. The method includes introducing the biological entity into the first fluid chamber of a device in accordance with the fourth aspect of the invention. A first (reference) electrical signal that is associated with a first status of the biological entity is first obtained. The biological entity is then exposed to a condition that is suspected to be capable of changing its status. A second electrical signal that is associated with the status of the biological entity after exposure to the condition is taken, and for example analysed against the first electrical signal.

Noteworthy, the present invention is suitable for providing lateral patch clamp apertures and/or patch channels of a profile that is at least substantially elliptical or at least substantially circular. In some embodiments the respective profile is fully circular in shape. An advantage of round or circular apertures in patch clamp devices is the possibility of achieving high seal electrical resistances when a patch clamp on a sample biological entity is exerted through suction exerted through the patch aperture. Rounded apertures are known to be capable of providing seal resistances that are in the order of giga-ohms, thereby reducing background noise signals and thus enabling more accurate patch clamp measurements to be taken. Furthermore, as the present invention provides the ability to fabricate apertures with dimensions ranging from several micrometers to sub-micrometer levels, the device can be used on applications involving many types of biological samples other than cells such as bacteria, virus, protein, and DNA molecules. From the point of view of the fabrication of the device, there is a shorter turnaround due to the fewer steps involved, as only a shallow etch is required, so there is no need to etch through the substrate, unlike the planar patch clamping, thereby saving time in fabrication. The skilled artisan will further appreciate that the device of the invention allows a convenient packaging of the device by means of a capping layer which includes microfluidic input and output channels and ports, and scalability to achieve a high-density array suitable for large scale parallel testing, since the wall portions of the device that include channels can be formed to take little space and the profile of the channels formed in these wall portions can be defined lithographically, unlike diaphragms used in existing planar patch clamps.

The present invention is applicable to any type of small particle having a size in the range of several millimetres to less than about 1 micrometer. In this context, the term ‘particle’ includes both inorganic particles (such as silica microspheres and glass beads) and organic particles. The term ‘particle’ also includes biological entities, which in this context refers to biological material, including tissue fragments, sperms, individual cells of an organ or tissue, and subcellular structures within a cell; single cell organisms such as protozoans, bacteria cells and viruses, as well as multi-cell organisms. The term ‘biological entity’ is also used interchangeably with other equivalent terms, such as “bio-molecular body” or “sample biological entity”. Cells to which the invention can be applied generally encompass any type of cell that is voltage sensitive, or a cell that is able to undergo a change in its electrical potential, wherein the cell may be both an eukaryotic cell or a prokaryotic cell. Examples of a suitable eukaryotic cell include both a plant and an animal cell. Examples of suitable animal cells include, but are not limited to, cells in the nervous system such as astrocytes, oligodendrocytes, Schwann cells; autonomic neuron cells such as cholinergic neural cell, adrenergic neural cell, and peptidergic neural cell; sensory transducer cells such as olfactory cells, auditory cells, photoreceptors; hormone secreting cells such as somatotropes, lactotropes, thyrotropes, gonadotropes and corticotropes from the anterior pituitary glands, thyroid gland cells and adrenal gland cells; endocrine secretory epithelial cells such as mammary gland cells, lacrimal gland cells, ceruminous gland cells, eccrine sweat glands cells, and sebaceous gland cells; and other cells including osteoblasts, fibroblasts, blastomeres, hepatocytes, neuronal cells, oocytes, Chinese hamster ovary cells, blood cells such as erythrocytes, lymphocytes or monocytes, muscle cells such as myocytes, stem cells such as embryonic stem cells. A mammalian cell is an illustrative example of a cell being commonly used in the art in the screening of drugs. Other examples of an eukaryotic cell include yeast cells and protozoa. Examples of a plant cell include meristematic cells, parenchyma cells, collenchyma cells and sclerenchyma cells. Prokaryotic cells applicable in the invention include, for example, archaea cells and bacteria cells. The term “biological entity” additionally encompasses other types of biological material such as subcellular (intracellular) structures such as an organelle, a centrosome, structures of the cytoskeleton, a cell membrane, cytosol, a cell wall and fragments, derivatives, and mixtures thereof. Examples of organelles include, but are not limited to, the nucleus (including the nucleolus), the endoplasmic reticulum, a vesicle such as an endosome or phagosome, Golgi apparatus, mitochondrion, lysosome, peroxisome, a vacuole, a chloroplast, and fragments, derivatives, and mixtures thereof.

### BRIEF DESCRIPTION OF THE DRAWINGS

The invention will be better understood with reference to the detailed description when considered in conjunction with the non-limiting examples and the accompanying drawings.

FIG. 1A to FIG. 1C show cross-sectional views of microfluidic devices according to exemplary embodiments of the present invention. FIG. 1D shows a perspective view of a partitioning element having a single channel; the arrow on the lower right shows the lateral direction in which the lateral channel is being arranged in the partitioning element. FIG. 1E shows a scanning electron microscope photograph of a cross section of the single channel. FIG. 1F depicts a further embodiment of a device of the invention in cross-sectional view. FIG. 1G shows a further embodiment of a microfluidic device of the invention in a perspective view. FIGS. 1H to 1O show embodiments of the microfluidic device in top view.

FIG. 2A to 2E depict a simplified illustration of a method of fabricating the device of the invention.

FIG. 3A shows a scanning electron microscopy image of a further microfluidic device according to the invention. This embodiment of the device includes a device as shown in FIG. 1. In the device depicted in FIG. 3A the latter device is a partitioning element. FIG. 3B to FIG. 3D show close up views of the aperture of the channel of the partitioning element.

FIG. 4 depicts a schematic overview of an embodiment of each of two alternative methods of the present invention of forming a microfluidic device (partitioning element in the device shown in FIG. 3).

FIG. 5 depicts schematics of a patch clamp recording setup with FIG. 5A showing a classical setup using a glass micropipette, FIG. 5B showing a planar patch setup, and FIG. 5C showing a lateral patch clamp setup.

FIG. 6 depicts close up images of apertures of a channel obtained using the method shown in FIG. 4B. The channel shown in FIG. 6B has a tapered end portion.

FIG. 7A shows the tip of a pulled conventional patch clamp pipette. FIG. 7B and FIG. 7C show two embodiments of a channel that includes a conical portion contiguous to its aperture.

FIG. 8 shows scanning electron microscopy images of a microfluidic device produced according to the method depicted in FIG. 4B before (FIG. 8A) and after (FIG. 8B) a focussed ion beam cut along the line A-A′.

FIG. 9 depicts the use of a device according to the invention as a patch clamp chip together with an illustration of respective experimental results. The device used in FIG. 9A and FIG. 9B was formed based on a process as depicted in FIG. 4A. The device used in FIG. 9C was formed based on a process as depicted in FIG. 4B.

FIG. 10 shows a summary of the electrical data of devices obtained by the method depicted in FIG. 4A (FIG. 10A) and depicted in FIG. 4B (FIG. 10B) that were tested as patch clamp chips.

FIG. 11A shows a perspective view of a partitioning element having a plurality of channels; FIG. 11B shows a electron microscope photograph of a cross section of the plurality of channels.

FIG. 12 shows a top view of two embodiments of a device of the invention that includes a plurality of channels and a plurality of second fluid chambers. The device of FIG. 12A includes a plurality of first fluid chambers, each of which is connected to a respective second isolated fluid chamber via a channel. The device of FIG. 12B includes a single first fluid chamber fluidly connected to the plurality of second fluid chambers.

FIG. 13 shows microscope photographs showing the various stages of the filler member undergoing deformation in a fabrication method of the invention.

FIG. 14 shows examples of how a whole cell can be immobilised using a device according to the present invention. The cell can be immobilised on the aperture of a channel (FIG. 14A & FIG. 14C) in a fluid chamber, or inside a channel (FIG. 14B & FIG. 14D).

FIG. 15 shows a close up image of an embodiment of a device according to the invention that includes additional auxiliary side channels.

FIG. 16 depicts a sequence of images showing capturing an isolated cell at the aperture of the channel of a device of the invention via applying controlled suction through additional auxiliary side channels.

### DETAILED DESCRIPTION OF THE INVENTION

The microfluidic device according to the invention includes a base substrate having a recess defined therein. The recess is included in the base substrate. It is defined by two or more opposing lateral walls and a base wall. Depending on the configuration desired, the recess may span (e.g. laterally, diagonally etc.) the entire length/width of the base substrate (i.e. from one edge or side to another edge or side). The recess may also be located near one edge of the base substrate or, if desired, near the center of the base substrate. Such a position of the recess allows the deposition of other matter on a wall portion, generally a surface, of the base substrate that includes the recess. In certain embodiments, for example where it is desired that the recess spans the entire surface or length (e.g. from end to end) of the base substrate, the recess is bounded by a pair of lateral walls, which are typically in opposing relationship. In some embodiments the ends of the recess are lateral apertures, e.g. openings. In some embodiments the recess includes one end at an edge of the base substrate, where the end may include an aperture, while another end terminates at a location within the base substrate that differs from the edge thereof (e.g. at the middle portion). In such an embodiment the recess is bounded by a pair of opposing lateral walls, a base wall, and an additional lateral wall connecting the two opposing lateral walls. The respective other end of the recess, which may be arranged in opposing relationship to the additional lateral wall, includes or defines an aperture. In some embodiments the ends of the recess are entirely included within the base substrate. In such embodiments the recess may include two pairs of opposing lateral walls and a base wall. In some embodiments the recess includes more than two ends. In such embodiments the recess typically includes a branching.

The recess may have any suitable shape, such as being a cuboid (e.g. rectangular or square shaped) in which case the recess is in the shape of a trench, or alternatively a hemi-sphere or any other suitable irregular shape. Regardless of the shape, the recess has in some embodiments a depth of at least about 5 μm, or for some embodiments with large aperture diameters or for certain types of filling materials (see below), at least about 20 μm, including about 50 μm, such as from about 1 μm to about 50 μm, from about 2 μm to about 20 μm or from about 6 μm to 8 μm. Where a hemispherical shaped recess is formed in the base substrate, it is to be noted that the recess is then defined by a continuous wall. In this case, any two directly opposing end portions of the hemispherical walls of the recess may be considered to be the opposing lateral walls of the recess in accordance with the invention. The same applies to an irregularly shaped recess.

The base substrate may have one circumferential wall or a plurality of lateral walls. It may also have a base and a top wall. The microfluidic device of the invention further includes a filler member. The filler member may cover any area of any of these walls. Typically the filler member forms one continuous entity. As an illustrative example the base substrate may include a plurality of lateral walls and a top wall. The recess may be included in the top wall. In some of such embodiments the filler member covers a portion of the top wall, including the entire top wall. Accordingly, the filler member may cover any surface portion or portions of the base substrate. In one embodiment the filler member defines the entire surface of the base substrate.

The filler member is arranged such that at least a portion of it occupies the recess present in the base substrate. In some embodiments the filler member merely fills the recess. In other embodiments only a portion of the filler member is included in the recess. Thus, in such embodiments the filler member covers a part of the surface of the base substrate and extends continuously into the recess. In some embodiments the portion of the filler member that occupies the recess merely fills the recess to a certain extend, while the remaining portion of the recess is filled with a fluid, such as a gas.

The portion of the filler member included in the recess has defined therein one or more sub-surface channels. The channel(s) terminates in a first aperture, which may for instance serve as an inlet, and a second aperture, which may serve as an outlet. Both the first and the second aperture are included in a wall of the base substrate as defined above. As an illustrative example, the first aperture and the second aperture may be included in two different lateral walls of the base substrate. As another illustrative example the first and the second aperture may be included in two different wall portions of a circumferential wall of the base substrate. Accordingly, the first aperture and the second aperture are included in two lateral walls of the filler member, which are arranged in opposing relationship with each other. The term “opposing relationship” refers to the direction of matter that would flow through the recess and/or the channel, such as an axis of the channel. Accordingly, the two lateral walls of the filler member may be arranged in any angle with respect to each other, as long as the first and the second aperture are not facing the same direction. The first and the second lateral wall may for instance be inclined with respect to each other in an angle from 0 to 90°.

In some embodiments the first lateral wall and the second lateral wall of the filler member are orientated to be at least substantially perpendicular (also used interchangeably with the term ‘orthogonal’) to the opposing lateral walls of the recess. As noted above, the lateral apertures of the channel are included in lateral walls of the filler member. As an illustrative example a recess in the form of a trench may be included in the top wall of a cuboid base substrate. In such an embodiment the orientation of at least the first aperture, or the second aperture as well, formed on the lateral walls of the filler member is such that the plane of each aperture is at least substantially vertical, thereby achieving lateral apertures on the lateral walls of the filler member. By the term ‘substantially perpendicular’, it is meant that the angle between the plane of the opposing lateral walls of the filler member may be arranged not exactly at 90° to the plane of the opposing lateral walls defining the recess. The angle may deviate from 90°, as long as a part of the opening of the aperture is accessible horizontally.

In some embodiments the channel is defined by a circumferential wall. In one embodiment, the cross-section of this circumferential wall in at least a portion of the channel has an at least substantially circular or an at least substantially elliptical-shaped profile. The term ‘at least substantially circular’ as applied to the cross-sectional shape of the channel includes any form that covers a 360° angle at the opening and thus means that it may be perfectly circular, or it may be, for example, elliptical or oval in shape. As it may be desirable to achieve substantially circular apertures, fluid chambers, as described below, can be formed to coincide with this circular cross-sectional portion of the channel so that a circular aperture opening up into the fluid chamber is achieved. Furthermore, in some embodiments at least one of the apertures of the channel defines an area of at least substantially circular or elliptical shape. At least the first (e.g. inlet) aperture may for instance be at least substantially circular or elliptical in shape; in other embodiments, both the first aperture and the second (e.g. outlet) aperture may be at least substantially circular or elliptical in shape. In some embodiments the first aperture is elliptical, or at least substantially elliptical, in shape, while the second aperture is circular, or at least substantially circular in shape. In some embodiments the first aperture and the second aperture are circular, or at least substantially circular in shape.

Depending on the application for which the device is intended, the dimensions of the first and the second apertures may be varied. For example, for patch clamp applications, the aperture may be adapted to be sufficiently small to achieve an effective seal on the surface of a sample particle or biological entity through the application of a suction force. If the sample biological entity is a human egg cell having a diameter of about 100 μm, the aperture that is used for performing the patch clamp can have a diameter from about 0.1 μm to about 10 μm, such as from about 1 μm to about 3 μm. For smaller cells such as red blood cells, which typically have a diameter of about 5 μm, the aperture can have a smaller diameter of from about 0.1 to about 1 μm, if necessary. The diameter of the first and the second apertures may be the same or different. In patch clamp applications, it is not necessary for both apertures to be circular in shape but it is only necessary for the inlet aperture to be circular to achieve an effective patch clamp. An aperture serving as an outlet, for instance the second aperture, may therefore assume any other shape, since it is not used for patch clamping. Where only one aperture is at least substantially circular or elliptical in shape, this aperture may be arranged to face the fluid chamber that is to be used to accommodate a sample particle (see below), such as the first fluid chamber. In embodiments in which both the first and the corresponding second apertures are at least substantially circular or elliptical in shape, either aperture may serve as the inlet for patch clamping the sample biological entity.

The channel connecting the first aperture to the second aperture may be of a profile of any suitable cross-sectional shape, for example the shape of a circle, a semi-circle, an egg, letters V or U, a triangle, a rectangle, a square, a pentagon a hexagon, a heptagon, an octagon or any other oligoedron. In some embodiments the profile of the channel is of the same shape throughout the entire length of the channel. In other embodiments, the profile of the channel changes its shape, for instance gradually or stepwise. An aperture of the channel, including the first and the second aperture, defines an area, which may likewise be of any shape. Any aperture of the channel, such as the first aperture and the second aperture, may for instance define an area with the shape of a circle, a semi-circle, an egg, letters V or U, a triangle, a rectangle, a square, or any oligoedron. In some embodiments at least one of the first and the second aperture of the channel define an area of at least substantially circular or at least substantially elliptical shape. In some embodiments the channel has the same cross-sectional shape as one or both the first and the second aperture. In some embodiments the area defined by the first aperture or the second aperture has similar dimensions as the profile of the cross section of the channel. In some embodiments the area defined by the first aperture or the second aperture is of a maximal size in terms of its width selected in the range from about 0.1 μm to about 10 μm. In some embodiments the area defined by both the first and the second apertures is of a maximal size in terms of its width from about 0.1 μm to about 10 μm.

As already noted above, in some embodiments the channel has a maximal size in terms of its width that varies along the length of the channel. Respective variations of the maximal size in terms of the channel width may be designed anywhere in the channel and include a progressive change in one or more steps or a continuous change. Such changes include, but are not limited to a constriction, a dilatation, a protrusion or a vaulting. In some embodiments the maximal of the channel size in terms of its width changes in vicinity to an aperture. The channel may for instance be defined by a circumferential wall. Such a circumferential wall of the channel may have a portion contiguous to an aperture, such as a first aperture, a second aperture or both a first and a second aperture. This portion of the circumferential wall may be conical along its length in that the size of the circumferential wall in terms of its width decreases toward the respective aperture. Noteworthy, such a portion of the circumferential wall of a channel resembles a pulled conventional patch clamp pipette with a narrow mouth and widening body. Such conical channel configuration typically reduces the “access resistance” as compared to a channel with a constant size in terms of its width. Those skilled in the art will appreciate that such a configuration is important in low-noise recording. Accordingly, it may be desired to select a respective channel design for patch clamp applications of the device of the invention.

In some embodiments the channel is arranged laterally within the filler member, i.e. within a horizontal plane of the base substrate. This does not preclude the possibility that sections of the channel are arranged to slope upwards or downwards within the filler member. In some embodiments the channel has a maximal size in terms of its width, for instance a diameter, which is from about 0.5 μm to about 20 μm, such as from about 0.5 μm to about 10 μm. In some embodiments the channel the channel has a maximal size in terms of its width from about 5 μm to about 20 μm, such as from about 5 μm to about 10 μm. The longitudinal or axial length of the channel maybe orientated to be in alignment with the length or width of the recess. The channel may be of any desired length. In some embodiments, the channel has a length from about 1 μm to about 250 μm, such as about 1 μm to about 100 μm, or about 1 μm to about 50 μm.

The base substrate may include or be of any desired material. As an example the base substrate may include a metal, a metalloid, ceramics, a metal oxide, a metalloid oxide or oxide ceramics. Examples of suitable metalloids include, but are not limited to silicon, boron, germanium, antimony and composites thereof. Examples of suitable metals include, but are not limited to iron (e.g. steel), aluminium, gold, silver, chromium, tin, copper, titanium, zinc, aluminium, lead and composites thereof. A respective oxide of any of these metalloids and metals may be used as a metalloid oxide or metal oxide respectively. As an illustrative example, the base substrate may be of quartz or a glass. The term “glass” as used herein broadly refers to an amorphous solid material, which in terms of thermodynamics corresponds to a sub-cooled liquid. Accordingly, a glass is obtained by melting a substance and rapidly cooling it below its melting point. Upon doing so, no crystalline structure is formed. The term “glass” in particular refers to molten inorganic material selected from the group of silicon dioxide, sodium carbonate, potassium carbonate, manganese dioxide and metal oxides that has solidified. Examples of ceramics include, but are not limited to, silicate ceramics, oxide ceramics, carbide ceramics or nitride ceramics.

In some embodiments, the base substrate, which may serve as a partitioning element (see below), may include a material selected from the group consisting of any variety of silicon, germanium, silicon dioxide (such as quartz or glass), germanium oxide, aluminium oxide, silicon nitride, silicon carbide, metal and composites thereof. In some embodiments the base substrate is derived from a conventional silicon wafer/chip obtainable from silicon foundries, including, but not limited to, Czochralski (CZ) wafers, Float Zone (FZ) wafers, silicon epitaxial (SE) wafers and silicon on insulator (SOI) wafers. In some embodiments, the filler member includes a dielectric material, such as an insulator. Examples of a suitable dielectric material include, but are not limited to, spin-on-glass (SOG), phospho-silicate glass, boro-phospho-silicate glass, polysilicon, silicon carbide, silicon oxycarbide, silicon nitride and composites thereof.

As indicated above, the base substrate may include a circumferential wall or a lateral wall (see above). In some embodiments at least a portion of such a wall of the base substrate is at least substantially parallel to the plane defined by the first lateral wall of the filler member. In some embodiments a respective portion of a wall of the base substrate includes at least a portion of the first lateral wall of the filler member. In some embodiments the base substrate includes two lateral walls, for example on two opposing sides of the base substrate. At least a portion of a first of these two lateral walls may be at least substantially parallel to the plane defined by the first lateral wall of the filler member. At least a portion of a second of these two lateral walls may be at least substantially parallel to the plane defined by the second lateral wall of the filler member. In such embodiments with two lateral walls a portion of the second lateral wall of the base substrate includes at least a portion of the second lateral wall of the filler member.

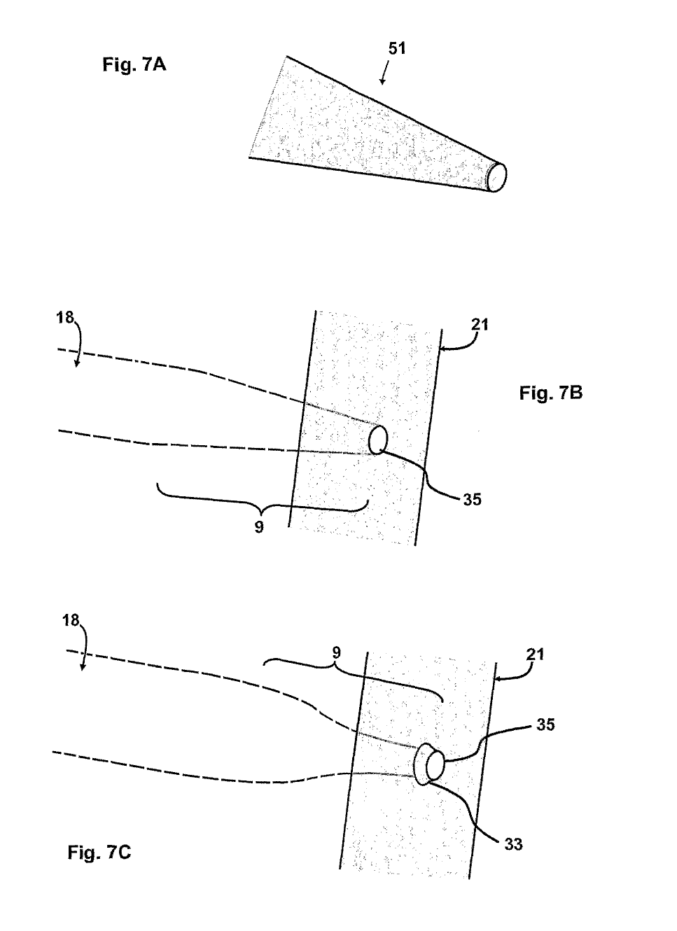

In some embodiments the first lateral wall of the filler member defines a wall portion of the base substrate. A respective wall portion of the base substrate may for instance be a circumferential wall or a lateral wall. In some embodiments the second lateral wall of the filler member also defines a wall portion of the base substrate. In some embodiments the wall portion defined by the first lateral wall of the filler member and the wall portion defined by the second lateral wall of the filler member are included in different lateral walls of the base substrate. A respective wall portion of a wall of the base substrate may be of any surface topology. It may for instance be a curved wall, a stepped wall or a straight wall. In some embodiments it may be of uniform topology, for example at least essentially flat or at least essentially bent at a uniform angle. Such a wall portion may also include areas of different surface topology. As an illustrative example, an area contiguous to the aperture of the channel may be of a three-dimensional structure that differs from the remaining surface of the lateral wall. In some embodiments the respective surface is arcuate in a plane that is perpendicular to the plane defined by the surface of the lateral wall. It may for instance define a convexity or a concavity. In the center of a respective convexity or concavity the aperture of the channel may be defined (see e.g. FIG. 7C).

In some embodiments a respective portion of a wall of the base substrate is included in a lateral recess of the base substrate. In some embodiments the wall portion of the base substrate defined by the lateral wall of the filler member includes a lateral recess. In such embodiments the lateral recess may include the first aperture or the second aperture of the channel. A respective lateral recess may be of any desired depth. It may for instance have a depth from about 0 to about 500 μm, from about 0 to about 250 μm or to about 100 μm, such as about 0 to about 25 μm, including from about 0.5 μm to about 15 μm, to about 50 μm or to about 250 μm.

In typical embodiments the lateral recess of the base substrate is inclined with respect to the recess that includes a portion of the filler member. In some embodiments the lateral recess is arranged at least essentially perpendicular to the recess of which at least a portion is occupied by the filler member. As an illustrative example the base substrate may have a lateral wall and a top wall. The recess that includes at least to a certain extend some part of the filler member may for instance be included in the top wall of the base substrate. The lateral recess may be included in the lateral wall of the base substrate.

The lateral recess may include a circumferential wall and an inlet (e.g. an opening), thereby defining a fluid chamber. Furthermore the lateral recess may also include a base. In some embodiments the base substrate may include two or more lateral recesses as described above. In some of these embodiments one lateral recess may include the first aperture and another lateral recess may include the second aperture of the channel. The base substrate may include two such lateral recesses, a first and a second lateral recess on two at least essentially opposing sides of the base substrate. The first lateral recess may include at least a portion of the first lateral wall of the filler member and the second lateral recess may include at least a portion of the second lateral wall of the filler member. In such embodiments the first lateral recess may for example define a first fluid chamber and the second lateral recess may define a second fluid chamber. The first fluid chamber may be in fluid communication with the second fluid chamber via the channel.

In some embodiments of a microfluidic device according to the present invention includes a plurality of channels. As an example, the portion of the filler member occupying the recess of the base substrate may include a plurality of channels. Each of these channels may include a first aperture and a second aperture. The first aperture may be included in a first lateral wall portion of the filler member and the second aperture may be included in a second lateral wall portion of the filler member. In some embodiments the first aperture and/or the second aperture may be included in a lateral wall of the microfluidic device, as described above. In some embodiments the plurality of channels is included in a common recess. In some embodiments some or all channels of the plurality of channels have the same maximal size in terms of their width. In some embodiments some or all channels of the plurality of channels have apertures that define areas of the same maximal size in terms of their width. In some embodiments one or more of the channels of the plurality of channels have a larger maximal size in terms of their width than other respective channels. In some embodiments where a respective microfluidic device is used for patch clamping such wider channels may be used as auxiliary channels as described below.

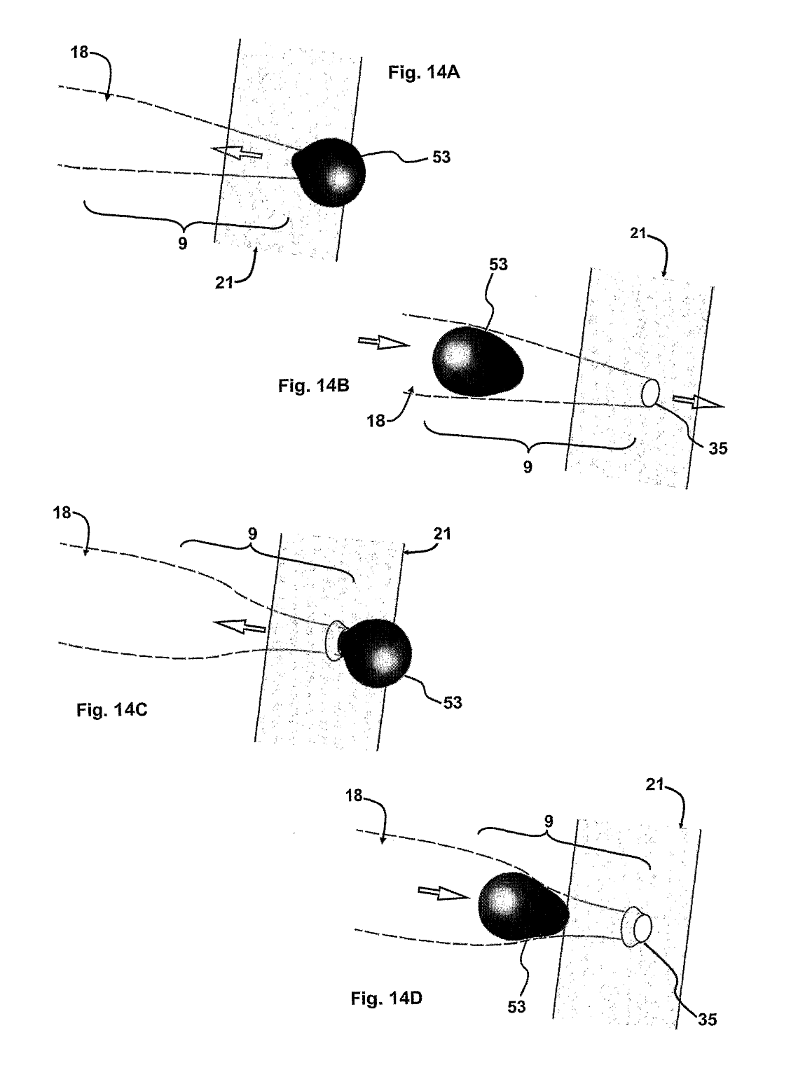

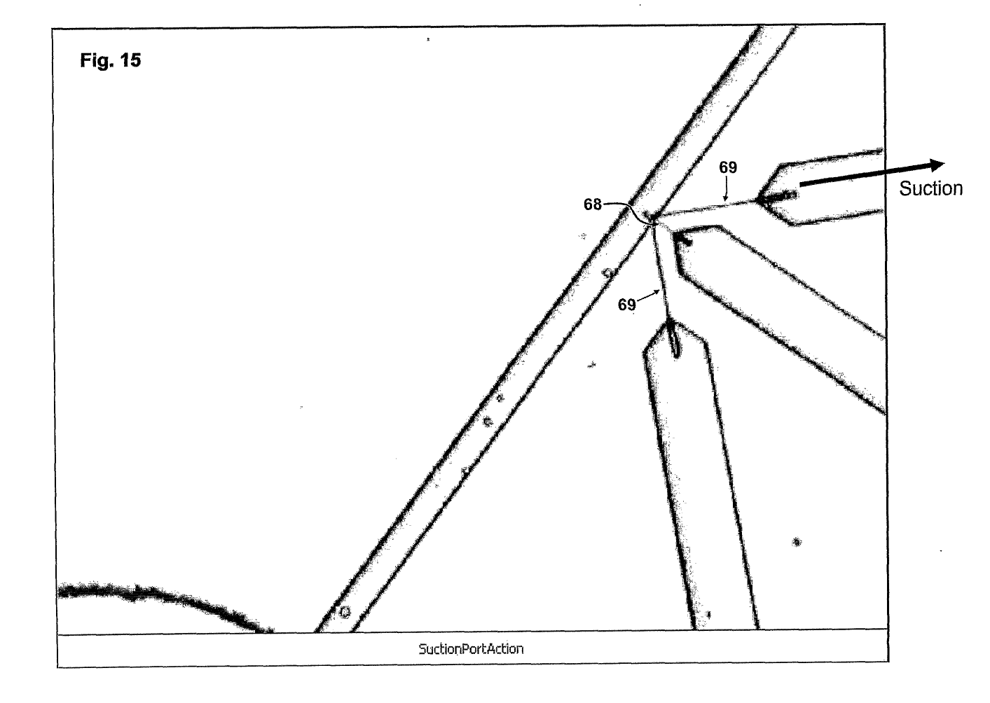

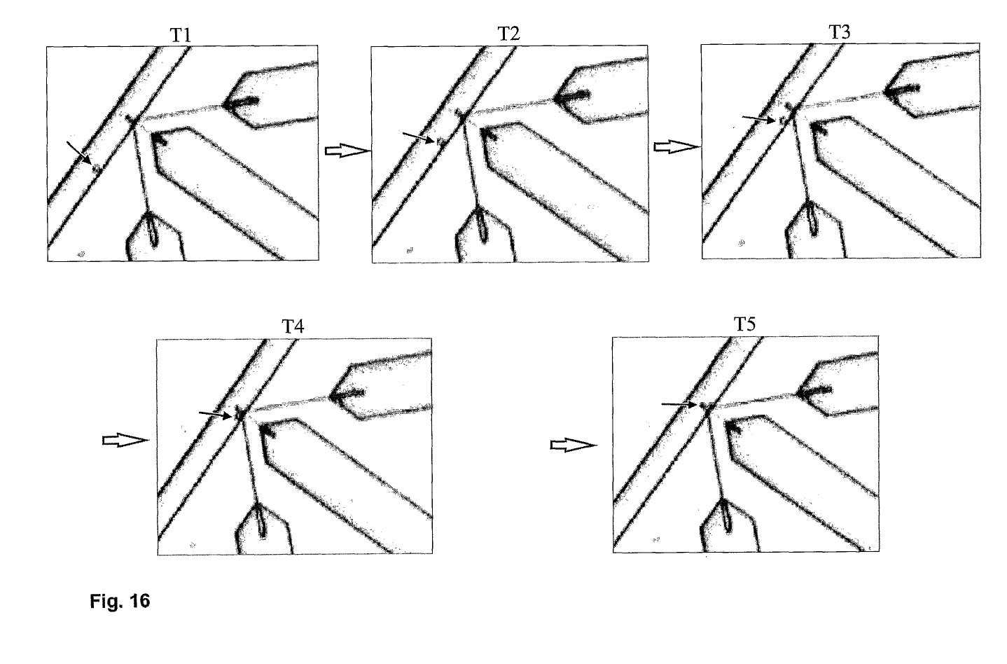

In some embodiments the microfluidic device includes one or more additional auxiliary channels. Typically such auxiliary channels are present in the base substrate rather than in the filler member. A respective auxiliary channel includes a first aperture and a second aperture. The first aperture is generally arranged on the same side of the microfluidic device as the first aperture of the channel as defined above, i.e. the channel present in the portion of the filler member occupying the recess. In some embodiments an auxiliary channel has a larger maximal size in terms of its width than a channel as defined above (present in the filler member). In some embodiments where a respective microfluidic device is used for patch clamping such an auxiliary channel may serve in positioning a biological entity at an aperture of a channel as defined above (see also below). An example of such an embodiment is depicted in FIGS. 14 and 15.

In some embodiments the first aperture of the auxiliary channel is arranged in a lateral wall portion of the base substrate. This lateral wall portion may include the first aperture of the channel present in the filler member. The first aperture of the auxiliary channel may be arranged in a recess of the lateral wall portion of the base substrate. Such a lateral recess may also include the first aperture of the channel present in the filler member. The first aperture of the auxiliary channel may in some embodiments be located in vicinity to the first aperture of the channel present in the filler member. The two apertures of the two channels may for instance be juxtaposed to each other.

In some embodiments the microfluidic device includes a plurality of recesses and a plurality of channels. In such embodiments the filler member may provide corresponding portions arranged such that they occupy at least a portion of each recess of the plurality of recesses. The plurality of channels may be arranged such that a channel is included in each of the corresponding portions of the filler member. In some embodiments several channels are included in one respective recess. In some embodiments the same number of channels is included in each recess. As an illustrative example a single channel may be included in each recess. In one of these embodiments each channel of the plurality of channels includes a first aperture and a second aperture. The first aperture may be included in a first lateral wall portion of the filler member, while the second aperture may be included in a second lateral wall portion of the filler member. The lateral walls of the respective portions of the filler member occupying at least a portion of the various recesses may have the same or different alignments and orientations with respect to each other. These lateral walls of the different portions of the filler member may for instance be inclined with respect to each other or define different planes, which may in some cases be parallel to each other. The apertures of the channels included in the plurality of recesses may likewise define areas that are inclined with respect to each other or that are arranged in different planes. In one embodiment all first lateral wall portions of the filler member that include the aperture of a respective channel are aligned so as to define a common plane.

Furthermore the lateral wall portions of the filler member, which occupy the various recesses of the plurality of recesses, may be arranged to be located in a common recess or in a number of recesses. In one embodiment each one of the respective lateral wall portions of the filler member is included in a separate recess. In some embodiments the plurality of first lateral wall portions of the filler member defines a wall portion of the base substrate. This said wall portion of the base substrate may be included in a lateral recess of the base substrate (cf. also above). In some embodiments the plurality of first lateral wall portions of the filler member defines a plurality of wall portions of the base substrate, Each wall portion of the plurality of wall portions may be included in a lateral recess of the base substrate, thereby defining a plurality of lateral recesses of the base substrate.

Taken in the context of a lateral patch clamp device, the aforementioned embodiments of the microfluidic device of the invention are directed to a partitioning element (hereinafter used interchangeably with the term ‘partitioning wall’) comprising the lateral channel with lateral apertures and which is used to separate two fluid chambers in a lateral patch clamp device. This partitioning element may be first fabricated and then assembled into a separate fluid chamber member to obtain the lateral patch clamp device. In other embodiments, the device of the invention may include a first fluid chamber that is separated from a second fluid chamber by the partitioning element, the first fluid chamber being in fluid communication with the second fluid chamber via the channel present in the filler member. For example, the first fluid chamber and the second fluid chamber may be monolithically defined in the base substrate at, respectively, the inlet aperture and the outlet aperture of the channel. In this manner, a lateral patch clamp comprising two fluid chambers connected through the channel in the filler member is realized.

In some embodiments, both the first fluid chamber and the second fluid chamber may be similar (identical) in shape, dimension and/or geometry. Alternatively, the first and the second fluid chambers may be different in shape, dimension and/or geometry. The first fluid chamber that is used for containing the sample biological entity may be a closed/isolated chamber or an open chamber fluidly connected to other fluid channels or a supply chamber. In one embodiment, the first fluid chamber is fluidly connected to a fluidic channel that is fluidly connected to a source supplying the sample. The second fluid chamber receives fluid from the first fluid chamber and may be fluidly connected to a drainage channel for discarding the sample.

Electrodes may be disposed in the first fluid chamber and the second fluid chamber for the purpose of taking electrical measurements between an upstream point and a downstream point of an immobilised particle or biological entity. Electrical measurements that can be taken include current flow (due to the flow of ions through the immobilised particle e.g. cell wall of an oocyte) as well as voltage potential, for instance. In the context of patch clamping applications, the electrode arranged in the upstream side of the immobilised particle maybe termed a reference electrode, and the electrode arranged in the downstream side of the immobilised particle may be termed a sensing electrode. More than one reference electrode and one sensing electrode can be positioned within the channel, e.g. close to the immobilised particle, functioning either for sensing purposes or for stimulating/electrocuting or moving the immobilised particle or for altering conditions in the fluid chambers. If it is desired to observe the response of the sample biological entity to electrical stimulation, additional electrodes can be arranged on the partitioning element, for example, in order to the sample biological entity, thereby stimulating it electrically. Auxiliary circuitry (e.g. electro-physiological measurement circuitry), either integrated into the device or provided by an external measurement system, may be connected to these electrodes.

If electrodes are not built into the device of the invention, such electrodes may be provided by an external measurement system, and may be arranged to be inserted into the fluid chamber via access ports. The device may also serve other purposes, notably for the filtering of particles. Filtering can serve a variety of purposes, including pre-concentrating a solution that includes a sample particle based on electrokinetic trapping (cf. Wang et al, Anal. Chem. (2005) 77, 4293-4299). Other examples of filtering applications include DNA sieving or the isolation of a virus sample, for instance. Filtering can be accomplished by placing a sample that includes particles that are to be sieved out into the first fluid chamber. By applying a suction force in the channel present in the filler member, particles smaller than the diameter of the narrowest section of the channel will enter into the channel and be discharged into the second fluid chamber. Particles larger than this diameter are trapped and remain within the first fluid chamber.

The device of the invention can be scaled up to process large quantities of the same or different samples simultaneously. For this purpose, the device may include a plurality of channels defined in the filler member, all of which are arranged in the portion of the filler member occupying a single recess. In an alternative embodiment, there may be a plurality of recesses defined in the base substrate and the filler member has corresponding portions thereof arranged in each recess. Each portion of the filler member occupying the recess may have defined therein a channel. A partitioning element comprising a plurality of channels may be used to separate a plurality of first and/or second fluid chambers, each of which is used to analyse a plurality of particles simultaneously.

In one embodiment, the device includes one common first fluid chamber and a plurality of second fluid chambers fluidly connected to the first fluid chamber via the plurality of channels in the partitioning element. In another embodiment, a plurality of first apertures is formed on the first surface of the partitioning element, and a plurality of second apertures is formed on the second surface of the substrate. Each first aperture of the plurality of first apertures is fluidly connected to a corresponding second aperture of said plurality of second apertures via a channel formed within the substrate, so that different samples can be placed within each individual first fluid chamber for simultaneous processing. In both embodiments, the second fluid chambers are isolated from each other to allow independent electrical recordings to be taken. To achieve this arrangement, the partitioning element may be bonded to the multi-well array such that each first aperture of the plurality of first apertures is in alignment with each individual first chamber of said plurality of first chambers. In a further embodiment, the device of the invention may include a plurality of partitioning elements, each of which is connected to a respective first fluid chamber constituting a multi-well array.

Where desired, the microfluidic device may include one or more elements for the formation of a concentration gradient. Such elements may for instance include a system of interconnected channels fluidly communicating with a plurality of reservoirs as described by Phil et al. (Anal. Chem. [2005] 77, 3897-3903). The formation of a concentration gradient of a compound to be tested may for example be desired in embodiments where the microfluidic device is to be used as a patch-clamp device. In such embodiments the creation of a concentration gradient may assist in the recording of dose-response curves, determining a K_{D} or K_{I} value, including e.g. an IC₅₀ value of a respective compound.

The microfluidic device according to the present invention may include any further element. As an illustrative example, the microfluidic device may include a temperature control element. As a further illustrative example, the microfluidic device may include cover members for covering a lateral recess of the device. Where a respective lateral recess defines a fluid chamber, a cover member, which may be removable, may serve in sealing the recess from the ambience. Sealing a respective recess that defines a fluid chamber may for example be desired to assist in the generation of a suction force in immobilising a biological entity on a channel aperture as described below.

The invention also provides a method of forming a microfluidic device as defined above. As an illustrative example, a respective method may, for instance, be a method of forming a lateral patch clamp aperture having patch apertures that are at least substantially circular in shape, and with circular cross-section diameter in the range of microns to nanometers. The method includes providing a base substrate. Where desired the roughness of a surface of the base substrate may be altered. As an illustrative example, a metal oxide or metalloid oxide surface, e.g. a silicon oxide surface, may be ground by means of sand paper (Ferrari, M., et al. Applied Physics Letters (2006) 88, 203125-1-203125-3). As a further illustrative example, the surface may be etched (cf. e.g. Cao, M. et al., J. Phys. Chem. B (2006) 110, 26, 13072-13075), for example using NaOH, KOH, a mixture of HF, HNO₃ and ethanol, a “buffered” HF solution containing NH₄F, or by ion bombardment using reactive ion etching.

The method further includes forming a recess on a surface of the base substrate. The recess can be formed by any conventional means, such as wet etching or dry etching. The dimensions of the recess can be varied according to the size of the particle to be analysed. In one embodiment, the width of the recess is from about 0.1 μm to about 20 μm, and the length is from about 1 μm to about 100 μm. In some embodiments the recess is open-ended in that it stretches up to at least one side of the base substrate.

In some embodiments a foundation layer is formed in the recess. Forming a respective foundation layer may for instance be carried out using any desired deposition method. A respective foundation layer may also be formed by surface treatment of the walls of the recess, such as for example thermal oxidation, which is commonly used in wafer production. The foundation layer may for instance form a film on the base wall and on the two opposing lateral walls of the recess. In one embodiment a foundation layer includes an insulating substance, such as thermal oxide, which may be a metal oxide or a metalloid oxide, such as silicon oxide, or any other oxide or nitride. In one embodiment the foundation layer is a structural layer such as polysilicon. A respective foundation layer is generally not deformable under conditions where the filler member is deformed (see below). It may furthermore be desired to carry out the formation of the foundation layer in a manner that does not significantly change or compromise the desired geometry and/or aspect ratio of the recess. As there are various deposition and oxidation methods available in the art, the skilled artisan can easily chose the most suitable method of forming a foundation layer, if desired.

The recess is filled with a deformable filling material, which may include a dielectric material. In embodiments where a foundation layer is formed in the recess (see above), filling the recess with the respective filling material is typically performed after forming such a foundation layer. Any filling material capable of deforming is suitable for this purpose. In one embodiment, the filling material includes various types of doped oxides and/or doped silicate glasses, typically having a sufficiently low glass transition material in order for deformation to take place at relatively low temperatures. The filling may be carried out for example by means of a deposition process, including a growth process. In some embodiments filling the recess is achieved by covering the base substrate with the filling material. Thereby the recess is filled at the same time. Examples of a suitable deposition process include, but are not limited to, plasma enhanced chemical vapour deposition, inductive coupled plasma enhanced chemical vapour deposition (ICP-CVD), low pressure chemical vapour deposition, flame hydrolysis deposition (FHD), physical vapour deposition (sputtering), epitaxy or coating. Examples of a suitable coating process include, but are not limited to, spin-coating or dip-coating.

The filling material, which will provide the filler member as described above, is deposited into the recess in such a way as to trap a void within the filling material, in particular the void is to be trapped in the portion of the filling material occupying the recess in the base substrate. In other words, the filling of the recess with a filling material includes depositing the filling material into the recess in a manner that causes the filling material to pinch together at the opening end of the recess, thereby forming or trapping a void in the filling material. The void may for example extends laterally through the filler member from one end of the recess to the opposing end of the recess. The void includes the gas in which the deposition is carried out, such as process/depositing gases or air.

As noted above, in some embodiments the recess is open-ended in that it stretches up to at least one side of the base substrate. When a respective recess is filled with a filling material, this may yield a void that has an end that is adjacent to the open-ended side of the recess. The void may for example extend through the filler member from one end of the recess to the opposing end of the recess and thereby end in immediate vicinity to an open ended side of the recess.

In some embodiments of the present method of the invention the device provided may include additional structures such as holes, recesses or channels. Measures may need to be taken to avoid the filling of respective structures with filling material where required. Where the device includes further channels intended to be used as auxiliary channels for positioning a cell for patch clamping (e.g. FIG. 16), these channels will typically be desired to be wider than a channel intended to be used for patch clamping. Any such auxiliary channel or other structure of the device may thus in some embodiments be selected to be of a width that prevents a filling with filling material. In other embodiments the properties of the filling material or the deposition conditions may be selected to prevent the filling of respective structures such as auxiliary channels.

After deposition (including growing) of the filling material has completed and a void is formed or trapped in the filler member, the filling material is subjected to conditions that will cause the filling material to deform, thereby forming a channel in the filling material. In general, the deformation procedure to form the void in the recess depends on various factors, such as the width-to-depth ratio of the recess, profile of the recess, deposition pressure of the filler, etc. For example, it is possible to form the void by non-conformal deposition of the filling material into the recess. The void can be reshaped into a circular structure so that a circular channel is realized in the trench. This may be done by re-flowing the filling material.

In one embodiment, the filling material reflow is achieved by thermal cycle. Since each material has a different glass transition temperature, different temperature cycles are required for the filling material to reflow and thus squeeze the trapped void. The thermal cycle also depends on the initial size of the void and the final dimension of the channel required. The larger the initial void or the smaller the final desired channel cross-section, the higher the temperature and/or the longer the duration of the thermal cycle required. Heating duration can vary from a few minutes to few hours. The time required for heating the filling material in order to deform it sufficiently to obtain a channel with a cross section of an at least substantially circular or an at least substantially elliptically-shaped profile (including an aperture that defines an area of at least substantially circular or at least substantially elliptical shape) is therefore variable and depends on the initial void dimension, deposition conditions, heating temperature, heating pressure and final dimension of the aperture.

In one embodiment, the filling material is heated above its glass transition temperature, but below the melting point in order to bring about the deformation of the filling material. If doped silicate glasses are used as filling material, temperature range at which heating is carried out may be from about 800° C. to about 1200° C. for time periods from about 30 seconds, including from about one or two minutes, to several hours, such as up to about 3 hours or up to about one hour. In some embodiments the time period is from about 30 seconds to about 30 minutes, including up to about 10 minutes, e.g. about 240 seconds. The pressure at which heating takes place may be at sub-atmospheric to atmospheric pressure (which is around 760 Torr), depending on the heating temperature. The pressure may for example be selected in the range from about 3 Torr to about 760 Torr, such as about 50 Torr to about 760 Torr or about 200 Torr to about 760 Torr, including for instance in the range from about 3 Torr to about 50 Torr or from about 3 Torr to about 200 Torr.

In embodiments where the recess is open-ended a void may be formed that has an end adjacent to the open-ended side of the recess (see above). Upon subjecting the filling material to a condition that causes it to deform, a channel with a circumferential wall may then be allowed to be formed from the void. Using the method of the present invention will in particular allow forming a channel that ends in a portion adjacent to the open-ended side of the recess. This portion of the channel is conical in that along its length the size of the circumferential wall in terms of its width decreases toward the open-ended side of the recess.

Auxiliary structures may be formed around the channel, including fluid chambers, microfluidic channels, ports, and electrical circuitry may be integrated with the device. The formation of such structures is within the knowledge of the skilled person, and may be carried out, for example, via a combination of etching and deposition procedures.

The method of the invention may further include the formation of a further lateral recess of the base substrate. As already described above, such a recess may include a portion of a lateral wall of the portion of the filler member that occupies the recess. The formation of a further lateral recess may include the formation of an aperture of the channel defined in the portion of the filler member occupying the recess. The formation of a respective lateral recess of the base substrate may be achieved by any conventional lithographic or etching techniques including dry-etching or wet-etching, or by mechanical means.

In some embodiments the recess is formed on a surface of the base substrate, such that it is formed to be open-ended in that it stretches up into one or more sides of the base substrate. The recess may for instance span an entire wall, such as a top wall of the base substrate. Forming the recess and covering the base substrate is then carried out as described above. In some embodiments the base substrate is covered in such a way that a portion of a wall into which the open-ended recess stretches is covered with the filling material. The filling member covering this wall portion may form a continuous filling member that includes the portion of the filling member that is included in the recess. Accordingly, during this embodiment of the method of the invention a channel can be formed that ends directly at a side of the partitioning element. In this embodiment forming the aperture of the channel requires merely the removal of a portion of the filling member that covers the respective side wall without the requirement of removing any matter of the base substrate itself.

In some embodiments the microfluidic device of the invention, and accordingly the device formed according to the present method of the invention, the base substrate is of a material that is less deformable than the material of the filler member, e.g. under conditions of elevated temperature and/or reduced pressure (see above). In such embodiments, gentler processes can be employed, where it is merely required to remove a part, e.g. a portion of a surface layer, of the filler member rather than a portion of the base substrate. As an illustrative example, dry etching may be required to remove a portion of the base substrate, while a mild wet etching may be sufficient to remove a portion of the filler member. Dry etching may produce fairly rough surfaces. By avoiding such an etching process an extremely smooth filler member (e.g. glass) surface can be formed. A smooth surface may be desirable for certain applications, in particular for patch clamping, where it minimises the risk of mechanical damage to the delicate cell membrane. A smooth surface is furthermore advantageous for tight seal formation with a cell membrane. Wet etching may for example be performed using a Brønstedt base, such as metal hydroxide, or a Brønsted acid. As an illustrative example, where the filling material is titanium oxide, a nitric acid/hydrofluoric acid mixture, hydrochloric acid or concentrated sulfuric acid may be used to remove the same. Examples of suitable metal hydroxides include, but are not limited to sodium hydroxide, NaOH, potassium hydroxide, KOH, lithium hydroxide, LiOH, and calcium hydroxide, CaOH. An accurate removal of a portion of the filler member by means of wet etching can for instance be achieved by dipping the surface of the substrate in an etching solution and removing it within in a couple of seconds.

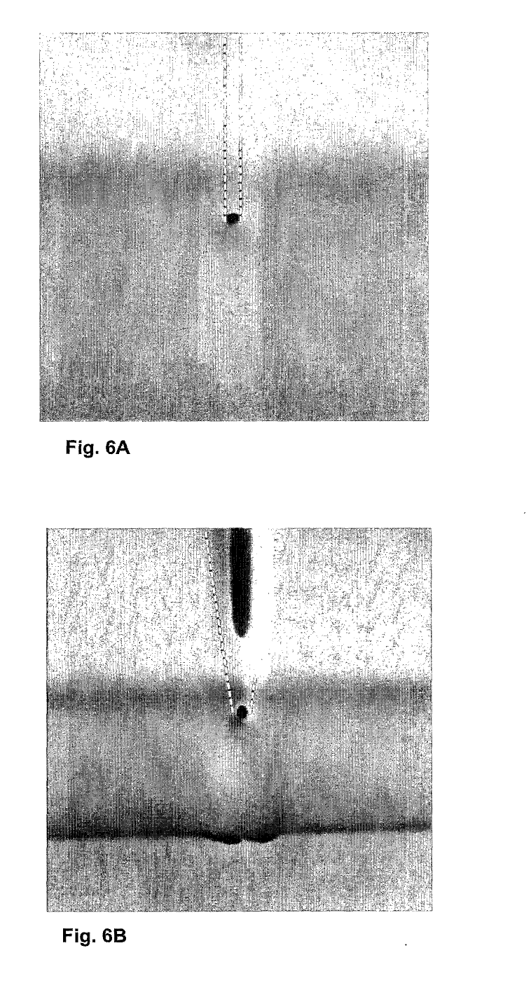

In some of these embodiments of the invention the method yields a three-dimensional surface in the form of a convexity or a concavity, such as a bulge, a dent, a ledge, an extrusion, a step and any combination thereof. A respective convexity may resemble the tip of a pipette. In some of these embodiments the method yields a portion of the channel contiguous to the aperture that is conical in that its width decreases toward the aperture as described above (see above and e.g. FIG. 6B, 7B or 7C). Those skilled in the art will appreciate that for e.g. patch clamp applications such a 3-D surface provides a contact surface suitable for adequate grip to the cell membrane. In particular it assists in the formation of a tight seal with the cell membrane. In this regard others have in the meantime observed that depositing undoped silicon dioxide on a patch-on-a-chip device, which already contained channels, yields an hourglass shape of the channel (Sordel, T., et al., Journal of Biotechnology (2006) 125, 142-154). Albeit obtaining a rough surface, these authors observed an improved seal in patch-clamp experiments using the obtained chip (ibid.).

In some embodiments the aperture of a channel that ends directly at a side of the base substrate may be formed by laser radiation. Laser radiation may for example be focussed on the respective area of the surface of the device as described in US patent application 2006/0003145. The use of laser radiation may for instance be desired in embodiments where an aperture that defines a small area, when compared to the width of the channel, is to be formed. As a further example, where the filling material includes a silicon oxide based material, such as quartz or glass, including e.g. borosilicate glass, an aperture may be formed by single ion track etching as described by Fertig et al. (Receptors & Channels [2003] 9, 1, 29-40; Appl. Phys. Lett. [2002] 81, 25, 4865-4867). Using hydrofluoridic acid, this method has been found to yield a tapered channel portion contiguous to the aperture as the ion track gets widened in hydrofluoridic acid. Thus a channel portion contiguous to the aperture may be formed that widens toward the aperture, i.e. the circumferential wall of which increases in terms of its width toward the aperture.

Forming a recess that stretches up to a side of the base substrate avoids the requirement of subsequently forming a further, e.g. lateral, recess therein in order to obtain apertures of the channel(s). For certain applications a respective recess may nevertheless be desired. In this regard in some embodiments of the method of the invention two recesses, a first recess and a second recess are formed in a base substrate. The two surfaces, or surface portions, on which the first and the second recess are formed, differ from each other. In some embodiments the two surfaces or surface portions are included in different walls of the base substrate. The surface on which the first recess is formed may for instance be a surface of a top wall. The second recess may for instance be formed in a surface of a lateral wall of the base substrate. In some embodiments at least a part of the surface or surface portion, on which the second recess is formed, is at least substantially perpendicular to the surface on which the first recess is formed.

The first and the second recess may be formed in any desired order. In some embodiments the first recess is formed before the second recess. In some embodiments other processes used in the invention are carried out between forming the first recess and the second recess. For instance the first recess may be filled with a filling material (see above), before a second recess is formed. As a further example an additional structural layer (such as a foundation layer as described above) may be formed on the base substrate before depositing the filling material. Such a structural layer is typically formed before forming a recess. The second recess may then be formed in such an additional structural layer. In other embodiments the first and the second recess are formed together, including concurrently. In one embodiment a first recess is formed in the base substrate. Thereafter a part of the base substrate, including the entire surface of the base substrate, is covered with filling material or an additional structural layer. Subsequently a second recess is formed in the filling material/additional structural layer. In case of using filling material this may, for example, be performed after a channel has been formed in the first recess. Such a second recess may be formed either to be included entirely in the filling material or an additional structural layer deposited on the base substrate, or the recess can cut through the filling material/additional structural layer and penetrate into the base substrate. Forming a respective second recess may follow the method already described above, after a filling material/additional structural layer has been deposited on the base substrate. In such an embodiment it may be desired to use a filling material, or material for an additional structural layer respectively, that does not easily deform under the conditions applied for forming the second recess. Such a layer could be silicon, polysilicon, or a derivative of the base substrate.

Reverting to the first recess, the first recess is formed on one of the surfaces of the base substrate as described above. This may include etching a surface of the base substrate. The recess may be formed to be open-ended in that it stretches up to at least one side of the base substrate. It may also stretch to two or more (e.g. where the recess is branched) sides of the base substrate. The first recess is in some embodiments formed before the second recess is formed. In one embodiment the first recess is filled with the filling material after the second recess is formed. In this embodiment the base substrate is therefore prestructured before any filling material is disposed thereon. As an illustrative example the base substrate may be prestructured by first forming a first recess on a top surface of the base substrate. Thereafter prestructuring may be continued by forming one or more second recesses, which may be lateral recesses.

Filling the first recess with the filling material may again be carried out by covering the base substrate with a filling material as described above. Thereby the first recess may at least partially be filled with the filling material (see above). At the same time at least a portion of the surface of the second recess may be covered with the filling material.

The second recess may be formed according to any desired protocol, including lithographic or etching techniques (see also above) or by mechanical means. The second recess may be formed such that the first recess disembogues therein. The second recess may in some embodiments be filled only partly with the filling material. In such embodiments the second recess may for example define a fluid chamber after the filling member has been disposed onto the base substrate. In embodiments where the second recess is formed to disembogue into the first recess, it may disembogue into the respective surface, or portion of the surface thereof, which is covered with the filling material. This surface portion may be a continuous portion The filling material covering this wall portion may then form a continuous portion together with the filling material included in the second recess.

This embodiment of the method of the invention may further include removing filling material that covers the surface portion, under which the end of the channel is arranged. Filling material may be removed until the end of the channel is exposed and a channel aperture is formed. This surface portion may be included in a lateral wall of the microfluidic device formed. In some embodiments this aspect of the present method includes removing at least essentially the filling material that covers the surface or portion of the surface of the second recess. Removing the filling material may be carried out by means of etching, such as wet-etching or dry-etching. As noted above, this embodiment of the method of the invention allows wet-etching for the entire variety of matter that can be used as material of the base substrate, since only the filling material is removed. This allows the formation of a smooth surface with a respectively smooth channel aperture.

Another illustrative example of a process of removing filling material that the present aspect of the method allows, is chemical mechanical polishing, known in the art also by its abbreviation “CMP”, is widely used as a process for planarising surfaces, in particular in the manufacture of semiconductor and microelectronics devices. Typically, chemical mechanical polishing includes the use of a rotating disk. On the disk a polishing pad, for instance of polyurethane, is attached. To the polishing pad a slurry or an aqueous dispersion is provided, which contain abrasives. An aqueous dispersion may for example include polyvinylpyrrolidone, an oxidant, a protective film forming agent such as an alkylbenzenesulfonate, and abrasive grains, as described in European patent application EP 1 757 665. Where desired, a surfactant may also be added. A stainless steel ring (Fe₂O₃), sapphire (Al₂O₃) and barium carbonate (BaCO₃) have also been used successfully as soft abrasives (see e.g. Chen, C.-C. A. et al. Journal of Materials Processing Technology (2003) 140, 373-378). This technology is well known in the art. As an illustrative example US patent application 2007/0049183 describes a CMP apparatus and method, as well as a polishing pad conditioning method. A detailed review on the technology has for instance been given by Zantye et al. (Materials Science and Engineering (2004) R45, 89-220. The underlying mechanisms of this complex polishing system are believed to involve several factors such as abrasive action, corrosion, electrochemical processes and kinetics.

Forming a pre-structured base substrate with one or more lateral recesses allows the formation of a device that includes both one or more fluid chambers and one or more channels with a tapered portion contiguous to the respective aperture as described above. Such a method may for instance be desired for forming a device according to the invention that is intended to be used for patch clamping purposes, in particular in the context of screening. As indicated above, this embodiment of the invention makes any requirement of dry etching of the base substrate redundant. Such a requirement (made redundant by this embodiment) would at the same time include dry etching of the filler member, which may for instance be a thick glass layer. This process typically takes time and may generate rough surfaces not only on the base substrate, but also on the filler member (e.g. a glass surface). This embodiment of the invention thus allows the formation of particularly smooth surfaces. At the same time a filler member with substantial thickness at least at its portion surrounding a recess, such as a thick layer of glass, can be deposited in order to minimize capacitive coupling without the risk or concern of the aperture being clogged.

The partitioning element can be fabricated independently, and then assembled with other components to form a complete device. For example, the partitioning element may be fabricated in a silicon wafer, and the silicon surrounding the partitioning element is entirely etched away to leave behind only the partitioning element. Subsequently, the partitioning element is assembled into a correspondingly sized fluid chamber and firmly attached by various bonding methods like anodic bonding, glue bonding, UV bonding, etc. The partitioning element is orientated to separates the fluid chamber into 2 sections, wherein the channel fluidly connects one section to another. Alternatively, the first and second fluid chambers may be formed monolithically into the base substrate with the recess and the filler member arranged between the two fluid chambers.

Those skilled in the art will appreciate that using the method of the present invention the channel cross section dimensions can be predicted and controlled through careful selection of parameters for deforming the filling material used for forming the filler member. Additionally, the process is CMOS compatible and hence can be integrated with other silicon technologies to realize other device components like electrodes, reservoirs, etc. Channel fabrication cost is low as no specialized tools/processes like electron beam lithography, wafer bonding or laser ablation are required. If desired, channels of different dimensions can be obtained within a single device by varying dimensions of the recesses formed on the surface of the partitioning element. Hence, a single device can be used for analysing different sizes of cells/biological molecules. Furthermore, the channels can be easily formed in the partitioning element due to the ability of the channels to self-align during fabrication. Smooth oxide surface is retained so that side wall roughness is reduced and wafer bonding can be easily carried out.

The microfluidic device according to the third and fourth aspects of the invention includes a first fluid chamber for containing a sample to be tested, a second fluid chamber that is separated from the first fluid chamber by a partitioning element as already described above as a further microfluidic device of the invention. The channel in the partitioning element is orientated such that the first aperture faces the first fluid chamber and the second aperture faces the second fluid chamber, thereby fluidly connecting the first fluid chamber to the second fluid chamber.

This device according to the third and fourth aspects of the invention represents the general form of a complete microfluidic chip which can be deployed at the end-user level to collect samples for analysis. This embodiment may be obtained several ways as mentioned earlier, for example, by fabricating the partitioning element independently, and then assembling the partitioning element into a fluid chamber member, for example by bonding; or by forming a first and a second fluid chambers monolithically into the base substrate with the recess with the filler member arranged between the two fluid chambers.

Various modifications can be implemented to make the chip more durable for physical handling and transportation. For example, the device may be provided with a glass lid or a polymer lid, for example of polydimethylsiloxane (PDMS), polycarbonate or poly(meth)acrylate, to cover the top of the filler member and the base substrate, as well as the top of the fluid chambers for sealing purposes. The chip may also incorporate a port which is capable of receive a delivery needle for introducing a particle sample into the first fluid chamber. Arrays of fluid chambers may also be connected via a plurality of channels to enable massively parallel testing to be carried out (e.g. screenings can be carried out simultaneously to determine the effect of many substances on a particle type of cell). In a commercial useful implementation, the device may be used in conjunction with a measuring system which takes readings from the device and which additionally provides electrical sensing circuitry, suction force control, data collection means, for example a computer for storing time and frequency domain signals recorded from biological entities, as well as statistical analysis to decipher the test results. It can also include an optical module for add-on optical characterization.

In one embodiment, an electrical measurement device is connected to the first fluid chamber and the second fluid chamber for determining one or more electrical characteristics of a test particle. The electrical measurement device may include a pair of electrodes connected to a current or voltage measurement equipment and which may each be inserted into the first fluid chamber and the second fluid chamber from access ports.

A further aspect of the invention is directed to the use of the device of the invention for analysing the status of a biological entity, for instance as carried out in a typical patch clamp test. In general, the biosensor of the invention may be used in any application requiring electrophysiological measurements of biological entities such as cells. Such applications typically require contact between the biological entity being evaluated and a current-sensitive sensor, such as a transistor or a conventional micropipette patch clamp or the sensing electrodes placed within the first and the second fluid chambers. Common applications for the biosensor include the screening of drugs (e.g. electrophysiological determination of compound activity on ion channels, an important class of therapeutic drug targets, in cell membranes is studied) and studies into the characteristics of cells (studies on the mechanisms of microelectrode electroporation).

The method includes introducing a biological entity into the first fluid chamber of a device in accordance with any suitable embodiment of the invention, namely, in accordance with the third and fourth aspects of the invention or in accordance with embodiments in accordance with other aspects of the invention and which include a fluid chamber.

The method typically further includes introducing a fluid into the fluid chambers of a respective device. The choice of the fluid used will depend on the biological entity used. Often the fluid will be a liquid, such as an aqueous solution. In some embodiments the fluid includes an emulsion, suspension, a vesicle, colloidal material or composite material. In typical embodiments introducing a fluid into the fluid chambers includes filling at least one channel providing fluid communication between the fluid chambers with the respective fluid. In some embodiments each channel of the microfluidic device (including one channel or a plurality of channels, were present) is filled with the fluid.

A first (reference) electrical signal that is associated with a first status of the biological entity is recorded via sensing electrodes that are either integrated into the device or provided by an external measuring equipment. Thereafter, the biological entity is exposed to a condition or stimulus that is suspected to be capable of changing the status of the biological entity. Exposure to such a condition includes, but is not limited to, surrounding the biological entity with a chemical compound which is being evaluated for efficacy on the biological entity, in particular a chemical compound which has is suspected to be capable of modulating the ion channel behaviour on the biological entity; the term also includes electrically stimulating the biological entity. In some embodiments the first electrical signal is a continuous signal. The respective continuous signal may be recorded continuously for any desired period of time.

After exposure to the condition, a second electrical signal that is associated with the status of the biological entity after exposure to the condition is measured. In some embodiments the second electrical signal is a continuous signal. Measurements of the first and the second electrical signal prior to and after exposure to the condition may be carried out continuously, meaning that the electrical signals may be continuously monitored before the exposure to the condition, until after the biological entity exhibits the full extent of the effect of the condition on it.

In cell membrane studies, e.g. studies characterising membrane polarisation, or studies determining trans-membrane threshold potential for pore formation can be made by making a first measurement of the electrical signal of the environment upstream and downstream of the biological entity in order to determine the ion current flow through the biological entity. Subsequently, after having exposed the biological entity to a condition suspected of being capable of altering the status of the cell, a second measurement of the ion current is made and is compared to the first measurement. The difference between the first and the second measurement can be compared to existing literature to determine whether the status of the biological entity before and after exposure to the condition. For example, the second electrical signal may be compared against a known electrical signal that is known to correspond to a changed status; alternatively, the magnitude of the difference between the first and the second electrical signal may be compared to the pre-determined threshold electrical signal value. When the magnitude of the difference between the first and the second electrical signal is larger than the magnitude of the pre-determined threshold electrical signal value, the condition to which the biological entity is exposed is determined to be capable of changing its status. In some embodiments a plurality of second electrical signals is detected during a continuous measurement. In such embodiments any number of the second electrical signals may be compared to the first electrical signal. The plurality of second signals may also be screened for a signal of maximal intensity when compared to the first electrical signal.

Measurements of the first and/or second electrical signal may include measurements of electrical current passing through any type of transport structure located within or isolated from the region of the biological entity, e.g. cell, on which the suction force is applied. For such purposes any technique and/or protocol known in the art may be employed in the method of the present invention. As an illustrative example, a patch-clamp measurement may be carried to determine a transmembrane voltage. Such a measurement may include setting a holding voltage or command potential, for instance in the form of a ramp of a desired structure. In accordance with conventional patch clamp techniques, the measurement may for instance be carried out on an intact cell using the whole cell or cell attached approach, or on a fragment of a cell using the inside-out and outside-out approach. For this purpose the biological entity, e.g. the cell, may be ruptured in a method according to the present invention. Thereby it may be allowed to access the electrical properties of a transport structure included therein. In this respect, examples of a transport structure in a cell include, but are not limited to, any of the following structures located in a cell membrane: anion channels, cation channels, anion transporters (including anion exchange transporters), cation transporters (including cation exchange transporters), receptor proteins and binding proteins. Measurement of the first electrical signal may include measuring a reference electrical potential of the sample solution containing the biological entity, said electrical potential being measured from a reference electrode present at the top surface of the biosensor and which is in contact with the sample solution. As an illustrative example, current passing through an ion channel of a cell, a cell membrane part, an organelle, an organelle part or a sperm may be detected and quantified. As a further illustrative example, vesicle exocytosis from a cell may be measured using patch amperometry analogous to the protocols disclosed e.g. by Dernick et al. (Nature Methods [2005] 2, 9, 699-708).

The present method of the invention may include positioning the biological entity at an aperture of a channel as defined above, for instance by applying hydrostatic pressure or by a flow of the fluid included in the fluid chambers and/or channels. The channel is included in the partitioning element of the microfluidic device. The biological entity may be positioned on an aperture of a respective channel or inside a respective channel. The present method of the invention may furthermore include immobilising the biological entity. Generally, by immobilising the biological entity a respective channel is sealed so as to prevent diffusion or other movement (including flow) of ions therethrough. Thereby the free movement of ions between the first and the second chamber is restricted.

In one embodiment, positioning and immobilising the biological entity on the biosensor is performed by means of suction force that is generated at the first aperture of a channel as defined above or at the aperture of an auxiliary channel (e.g. FIG. 15), as well as any other suitable types of forces such as dielectrophoresis. Suction force may for example operate via an aperture of a respective additional auxiliary channel. As noted above, this aperture of the additional auxiliary channel may be juxtaposed to the first aperture of a channel as defined above (i.e. a channel comprised in a portion of the filler member of the partitioning element). Immobilising the biological entity at a desired location within the microfluidic device is typically performed by means of suction force generated through the respective channel, at, in, or on the aperture of which the biological entity is to be immobilised. When a sample fluid is placed in the first fluid chamber, any suction force that is for instance applied through a respective channel results in fluid being drawn through the channel. The fluid is then entering the first aperture and subsequently draining through the aperture downstream of the channel, such as a second aperture. In some embodiments, by applying a sufficiently strong suction force, the particle is drawn towards the first aperture of the channel included in the recess as defined above. By further applying a suction force via the channel included in the recess as defined above, eventually the particle becomes patched over the first aperture thereof, forming a seal over the edges of the aperture and thereby restricting the free flow of fluid and ions through the channel. This arrangement establishes a high electrical resistance seal over the aperture. This suction force can be generated by withdrawing fluid from the second fluid chamber by means of a syringe, for example. Suction force can also be generated via pump-driven suction of the sample solution containing the biological entity.

Thus in some embodiments of the present method of the invention a biological entity such as a cell is positioned and immobilised at the aperture of a channel of the microfluidic device as described above. Positioning the biological entity may in some embodiments be carried out using additional detection means that are able to define the exact position of the biological entity, for example optical means such as a microscope. For this purpose the size of the biological entity and the aperture dimensions generally ought to be selected in such a way that the area defined by the respective channel aperture is of a shape that prevents the biological entity from passing through the aperture. Typically the area defined by the channel aperture is in such embodiments of a smaller size in terms of its width than the minimal size in terms of its width of the biological entity in at least one orientation of the biological entity.

In some embodiments the respective channel aperture is included in a lateral side wall of the microfluidic device that defines a first fluid chamber, into which a sample solution that includes the biological entity has been introduced. As an illustrative example, the aperture may be the first aperture (see above), which faces the first fluid chamber. In embodiments, where the first aperture is included in a concavity (see above), the concavity may assist in positioning the biological entity. In embodiments where the first aperture is included in a convexity, positioning and immobilising the biological entity may resemble conventional patch-clamping using a micropipette (see also FIG. 7C).

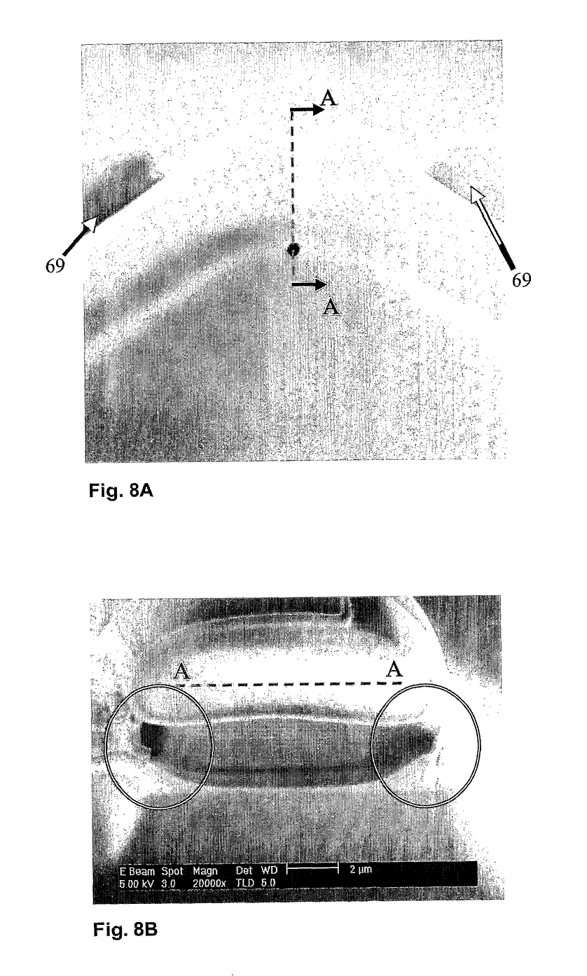

As a further example, the aperture may be the second aperture (see above), which faces the second fluid chamber. Typically, in such embodiments the channel includes a portion in which its profile changes in that the channel width in terms of its width decreases along the channel length. As an illustrative example, the channel may include a contraction, for instance located at some point inside the partitioning element. As a further example, the channel may include a conical (tapered) portion contiguous to the second aperture. The width of this conical portion gradually, e.g. continuously, decreases toward the second aperture (see e.g. FIG. 7B & FIG. 7C). In any of such embodiments the biological entity may be directed into the respective channel up to the contraction or the tapered portion. Accordingly, in such embodiments the biological entity is included in the respective channel and positioned before or in the contraction or the tapered portion. The biological entity may also be immobilised before or in the contraction or the tapered portion. In some embodiments where the channel includes a tapered end portion contiguous to the second aperture the biological entity may be positioned and immobilised before the second channel aperture. As an illustrative example, FIG. 8B depicts a channel with a width of about 2 μm and apertures of a width of about 1 μm. By forming a channel with only one tapered aperture (see also below in the examples) a respective channel with one aperture of a width of about 2 μm and one aperture of a width of about 1 μm can be provided. Such channel dimensions are suitable for entrapping prokaryotic cells with a diameter of ˜1 μm and certain organelles such as vacuoles and mitochondria. For other biological entities such as monocytes width a diameter of about 12-15 μm or eukaryotic nuclei with a diameter in the range of about 8 μm larger channel widths are required (see also below in the examples). Such an embodiment of the present method of the invention providing a channel with only one tapered end portion resembles the patch clamp technique described by Lepple-Wienhues and Ferlinz (Receptors & Channels [2003] 9, 1, 13-17). These authors have previously shown that immobilising cells within a micropipette yields extremely stable gigaseals at a high rate with high clamp quality (ibid.). The present method of the invention however dispenses with the previously faced challenge of introducing a biological entity into a glass capillary.

When using the device to carry out conventional patch clamp measurements on a biological entity, the sensing electrodes in the fluid chambers may for instance be used both to control the current (current clamp) or voltage potential (voltage clamp) in each fluid chamber and to measure the ionic current or membrane potential across the biological entity or the membrane potential across a membrane of the biomolecule, such as the membrane potential across the cell membrane of a cell. Measurements of the first electrical signal may include measuring an electrical current passing through at least one ion channel isolated within the region of a cell on which the suction force is applied.

If desired, optical analysis can be carried out to augment the electrical measurement analysis. For example, a visualization substance can be added to the first fluid chamber to assist a human operator to visually determine the status of the seal formed by the biological entity over the first aperture. The visualization substance can be a colour dye, such as ethidium bromide or disodium fluorescein, for example. If the pigment is seen travelling into the second fluid chamber, then the seal is not formed effectively and another attempt must be made to immobilise the biological entity over the aperture.

Apart from patch clamp applications, the device of the invention can also be used in various other applications such as capillary electrophoresis or DNA sieving. The device can also be used to immobilize or filtering any type of small particle over the laterally arranged aperture located on the filler member. For example, the device can be used for filtering and for trapping certain types of biological entity such as virii and pathogens. For filtering applications, the diameter of the inlet aperture can be in the sub-micron range. Application of suction force results in biological entity that are smaller than the aperture diameter to enter the aperture and then travel through the channel into the second fluid chamber, while large particles remain trapped within the first fluid chamber.

In embodiments where a device with a plurality of channels is used, multiple independent analyses, filtrations or other methods, for example patch-clamp measurements, may be carried out in parallel, including simultaneously. As an illustrative example, different patch-clamp protocols may be performed at the same time. As a further example, the plurality of channels may disembogue into a plurality of fluid chambers. In such embodiments a selected number, such as one or higher, of biological entities may be positioned in each respective fluid chamber in, behind or above an aperture of a channel. By providing different conditions, such as compounds present or concentrations of compounds present, in a selected number of the respective chambers, various parameters of interest may be analysed in parallel.

The present method of the invention, or any part thereof, may be carried out both manually and in an automated way. A device according to the present invention may in some embodiments be integrated into an apparatus for automated electrophysiological screening, for example in ion channel drug discovery. The present method of the invention may in such embodiments be used in primary screening as well as in secondary and safety screening of ion channel modulators. Besides e.g. primary compound screening, hit validation and lead optimisation, the present method may also be used in target discovery, target validation and assay development, both alone and in combination with other established methods, thereby shortening each respective phase. An illustrative example of such a further method is the usage of FLIPRs (Fluorometric Imaging Plate Reader) in the detection of membrane potential changes or the concentration of intracellular ions. Any respective method may include the use of patch-clamp robots, which may partly or fully automate patch-clamp recordings, including selecting and positioning cells, gigaseal formation, obtaining whole-cell or perforated-cell configuration, drug application, and data acquisition. Embodiments of the present method of the invention may furthermore include technologies such as “population patch clamp” (PPC), in which a single voltage-clamp amplifier sums the whole-cell currents of multiple cells at once, each sealed to a separate aperture in a device as described above. Any automated data analysis procedure such as the computational method for measuring the human ether-a-go-go channel (hERG) in high throughput screening presented by Miu et al. (Miu, P., et al. J.A.L.A. (2006) 11, 4, 195-202) may be employed. A further suitable example of an automated data analysis procedure has briefly been demonstrated by Asmild et al. (Receptors and Channels (2003) 9, 1, 49-58).

Where desired, the device and method of the present invention may be designed to analyse the status of a biological entity, including a plant (including water plants such as algae), or an animal such as for example a nematode, an aquatic invertebrate or a crustacean, in vivo. A respective in-vivo method may include the use of a fluorescent label and fluorimetric detection to assist positioning, such as by means of a two-photon-excited fluorescence laser scanning microscope (see e.g. Komai, S., et al., Nature Protocols (2006) 1, 2, 648-653 for suitable experimental procedures).

It has been reported that for certain commercially available automated electrophysiology devices such as PatchXpress® (Molecular Devices Corp., Sunnyvale, Calif., U.S.A.) some particularly potent compounds may be assigned an incorrectly low IC₅₀ value (Guo, L., & Guthrie, H., J. Pharmacol. Toxicol. Meth. (2005) 52, 123-135). This may not only be due to non-specific binding of hydrophobic compounds to the materials selected for the automated device, but also to the small dimensions of chambers that can be used (ibid.). Where required, it may therefore be desired to assess and optimise the performance of any protocol employed with an automated device based on the device according to the present invention as described by these authors (ibid.).

In order that the invention may be readily understood and put into practical effect, particular embodiments will now be described by way of the following non-limiting examples.

### Exemplary Embodiments of the Invention

Exemplary embodiments of methods according to the invention as well as reactants and further processes that may be used are shown in the appending figures.

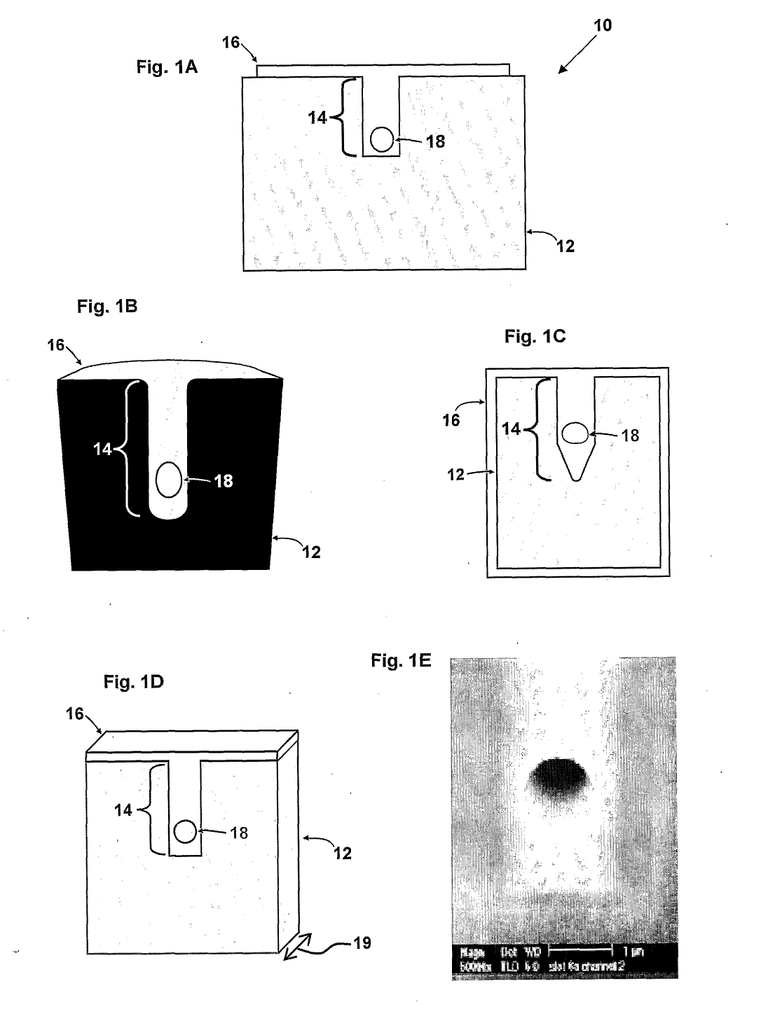

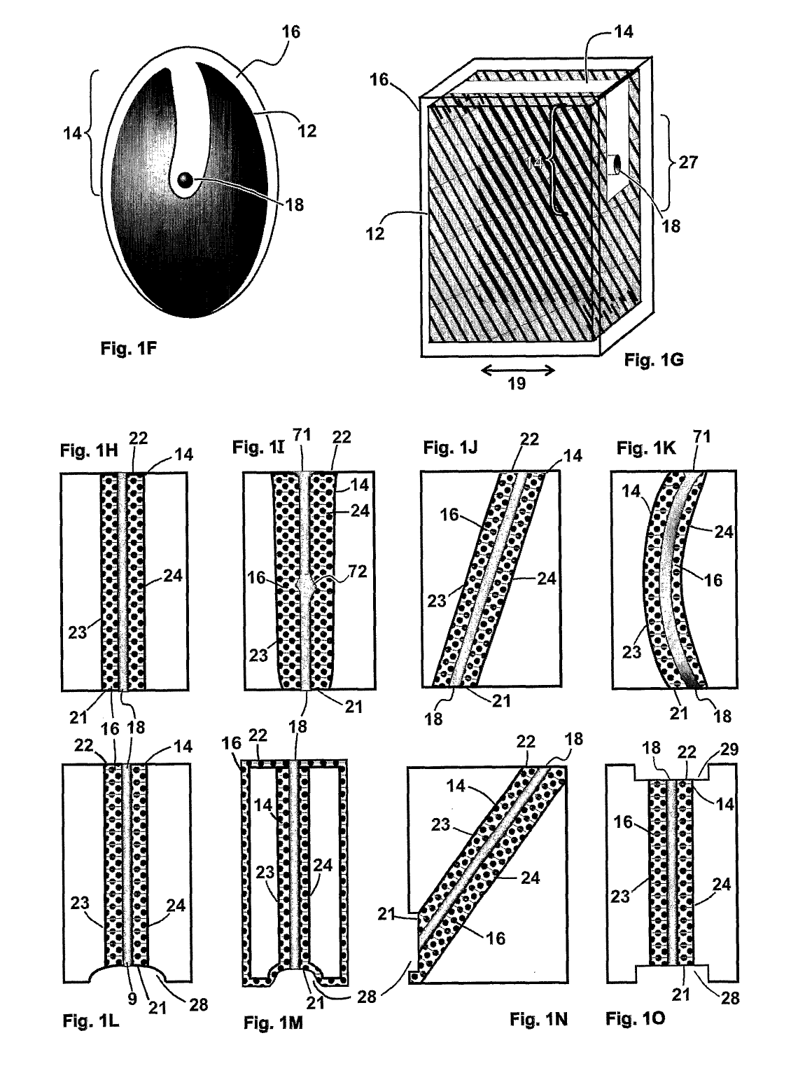

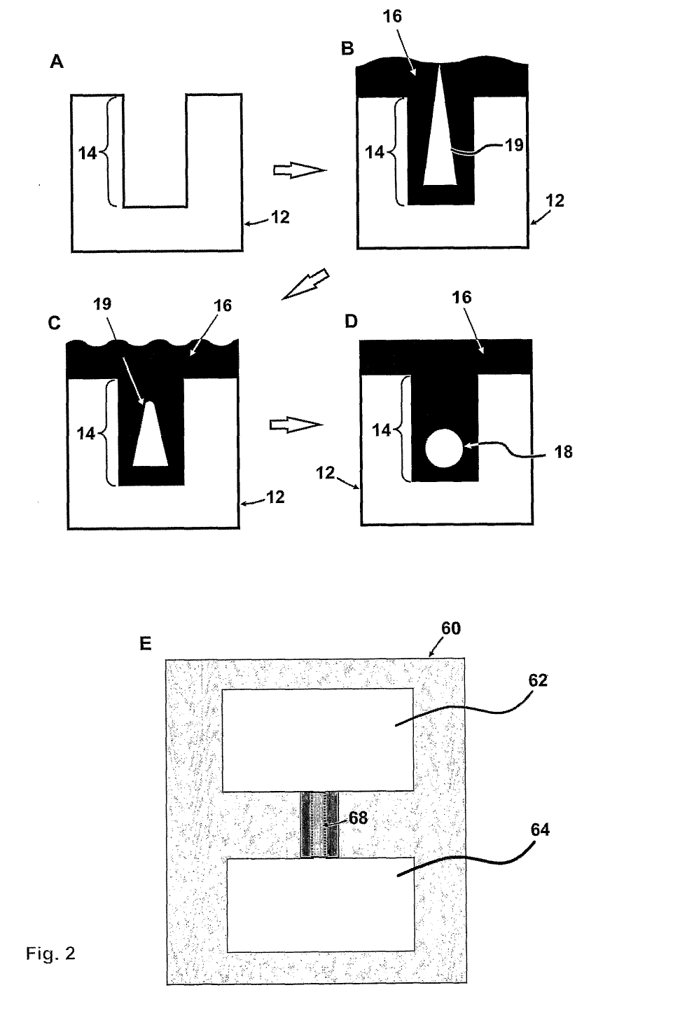

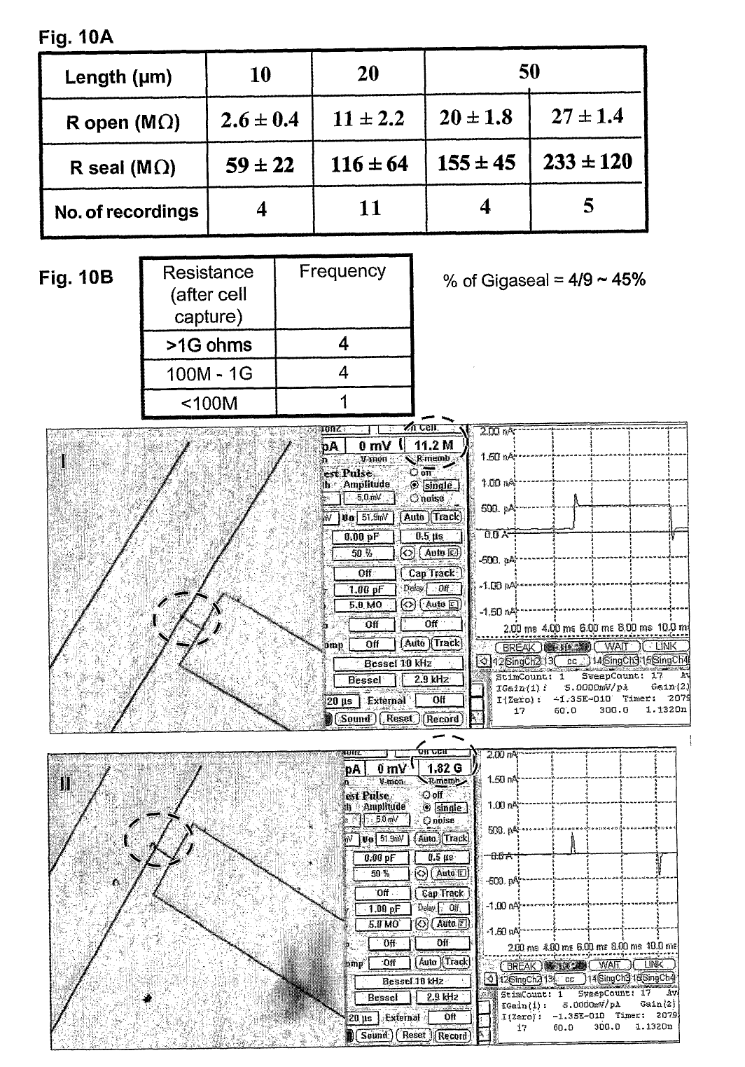

FIG. 1A to FIG. 1C depict cross-sections through a microfluidic device (10) (in a more general context also referred to as a partitioning element) according to a first embodiment of the present invention. The device (10) includes a base substrate (12) with a recess (14) formed on its top surface. A filler member (16) is arranged to cover at least a portion of the top surface of the base substrate (12) and occupies the recess (14). While in the microfluidic device depicted in FIG. 1 A the filler member (16) covers only a portion of the top surface, the microfluidic device depicted in FIG. 1B (and also in FIG. 1D, see below) includes a filler member (16) that covers the entire top surface of the device. In the device shown in FIG. 1C the filler member covers the entire surface of the microfluidic device. Any portion of the filler member (16) that covers a portion of a surface, such as the top surface, of the base substrate can be removed via etching or any other suitable means, if desired. A channel (18) is arranged to be present in the portion of the filler member (16) that is located in the recess (14). The terminal ends of the channel that include a first an a second aperture are arranged on a first and a second lateral wall of the filler member (16). The channel of the depicted embodiment is a straight channel. Accordingly, in the depicted cross-sectional view the profile of the channel is seen from the lateral wall of the filler member. The channel (18) is arranged within the recess (14), and the length of the channel is orientated to lie along the length of the recess. Given its orientation, the channel (18) is taken to be arranged laterally in the device (10). The recess (14) is defined by two opposing lateral walls and a base wall. The recess depicted in FIG. 1A (and also in FIG. 1D, see below) includes straight flat channel walls. In the microfluidic devices shown in FIG. 1 B and FIG. 1 C the base wall of the channel is of rounded profile in that it provides a concave surface. In the microfluidic device depicted in FIG. 1 C furthermore the two opposing lateral walls include inclined portions. FIG. 1D shows a perspective view of one embodiment of a microfluidic device (partitioning element) of the invention. The arrow symbol (19) on the lower right of FIG. 1D indicates the lateral direction with respect to the device (10). One aperture (which may either be an inlet or outlet) is formed on a first lateral side (facing the beholder) of the microfluidic device. Another aperture is formed on a lateral side that is arranged in opposing relationship with the first lateral wall of the filler member (averted from the beholder). Different surface topographies of the filler member, as shown in FIGS. 2B to 2D, may also be present in the microfluidic device after its fabrication. FIG. 1E shows a scanning electron microscope photograph of the channel opening of a microfluidic device according to the present invention. As can be seen in the figure, a substantially circular shaped aperture is typically obtained using the method of the present invention. FIG. 1F depicts a further embodiment of a device of the invention in cross-sectional view. Similar to the device shown in FIG. 1C, the filler member (16) essentially covers the entire surface of the device. The recess (14) in which the channel (18) is located, is defined by two opposing lateral walls and a base wall. One of the opposing lateral walls and the base wall have a surface of concave shape, while the second of the opposing lateral walls has a surface of convex shape. FIG. 1G shows a further embodiment of a microfluidic device of the invention in a perspective view. The arrow symbol (19) in on the bottom of FIG. 1G indicates the lateral direction with respect to the device. The filler member (16) covers the entire surface of the microfluidic device. The recess (14), depicted in white, in which the channel (18) is located is arranged on one side of the device, which is in the depicted orientation on the right hand side. The first lateral wall of the filler member (16) defines a wall portion (27), also depicted in white, of the base substrate (12). FIGS. 1H to 1O show embodiments of the microfluidic device (partitioning element) in top view. The respective microfluidic devices include a recess (14), which includes at least a portion of the filler member (16) (dotted), defined by two opposing lateral walls (23, 24) and a base wall (averted from the beholder). Two lateral walls (21, 22) of the filler member (14), arranged in opposing relationship, include the first and the second aperture, respectively, of the channel (18). The microfluidic devices depicted in FIGS. 1L, 1M and 1N furthermore include one lateral recess (28), the microfluidic device depicted in FIG. 10 two lateral recesses (28, 29). The recess (14) and likewise the channel (18) of the microfluidic device depicted in FIG. 1K are arcuate. The channel (18) of the microfluidic devices depicted in FIG. 1I and FIG. 1K have a conical portion (71) contiguous to an aperture of the channel. This portion (71) is widening toward the aperture. Likewise the channel (18) of the microfluidic device depicted in FIG. 1L has a conical portion (9) contiguous to an aperture of the channel. The size of this portion (9) in terms of its width is decreasing toward the aperture. Furthermore the channel (18) of the microfluidic devices depicted in FIG. 1I has a bulge (72), in which the channel has an expanded width. The microfluidic device depicted in FIG. 1M is entirely covered by the filler member (16).

FIG. 2 illustrates an exemplary method of forming a microfluidic device as illustrated in FIG. 1 according to the present invention. The method of forming a channel with a circumferential wall, the cross section of which has an at least substantially circular or an at least substantially elliptical-shaped profile, starts with etching a recess (14) on a silicon wafer (FIG. 2A), followed by partial filling of the recess (14) (FIG. 2B) with doped silicon oxide (such as PSG) as filling material, thereby forming the filler member (16). Partial filling refers to the incomplete filling of the recess (14) such that a void (19) in the shape of a through-channel is left behind in the doped silicon dioxide after the filling. Partial filling is carried out by simultaneously depositing the doped silicon oxide onto the lateral walls of the recess. By deforming or re-flowing the filling material, the shape of the cross section of the void (19) gradually approaches a circular shape, thereby realizing a circular channel profile in the recess. For this purpose, heat treatment is carried out over the glass transition temperature of the filling material. After heat treatment, the doped silicon oxide deforms and contracts (FIG. 2C) and pinches together at the opening of the recess to form a pinch portion, trapping a void beneath the pinched portion. After the void (19) is trapped, further heat treatment then deforms the doped silicon oxide further so that it reflows, thereby causing the void (19) to be gradually shaped into a channel that has a circumferential wall with a cross section of at least substantially circular or at least substantially elliptical-shaped profile (FIG. 2D). FIG. 2E shows a top view of a completed microfluidic device (60) that includes two fluid chambers (62, 64) and a channel (68) in the partitioning element at the dotted-line region.

The process parameters of temperature and pressure were varied to accomplish the formation of a circular channel. Six conditions that have been used are shown in Table 1 using borophosphosilicate glass (BPSG) and phosphosilicate glass (PSG) as filling material to form the filler member:

TABLE 1

| | Channel material & | Reflow temperature | |
|---|---|---|---|
| | Deposition Pressure | (° C.) | Time (min.) |
| 1. | BPSG at 50 Torr | 900 | 240 |
| 2. | BPSG at 50 Torr | 950 | 120 |
| 3. | BPSG at 50 Torr | 1000 | 40 |
| 4. | PSG at 3 Torr | 1050 | 120 |
| 5. | PSG at 3 Torr | 1100 | 45 |
| 6. | PSG at 3 Torr | 1150 | 30 |

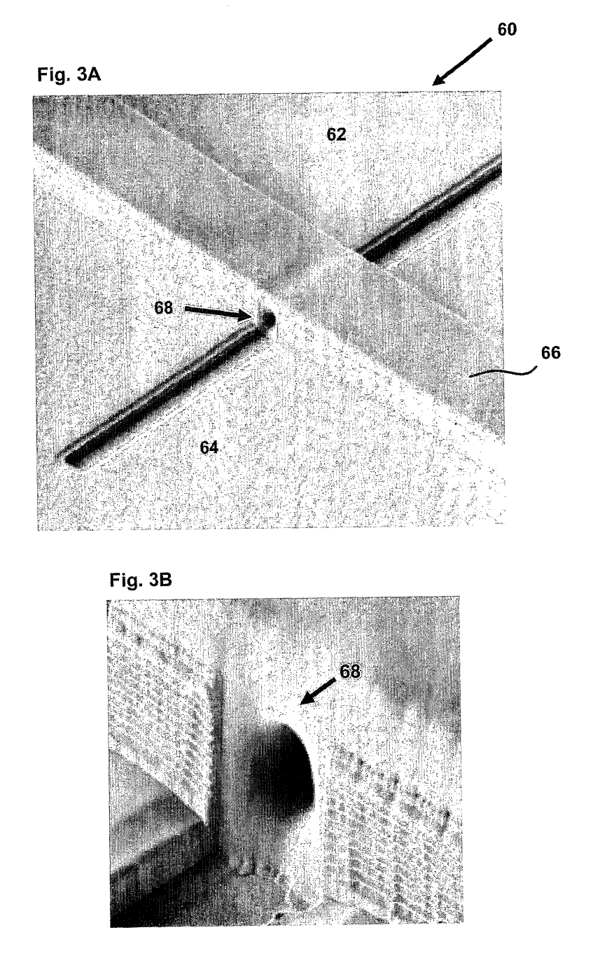

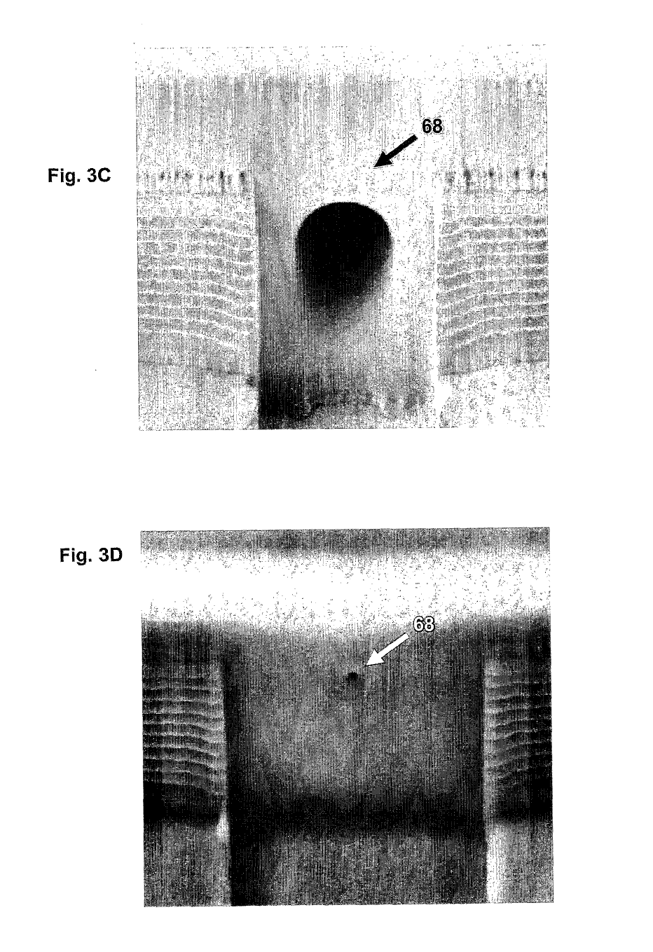

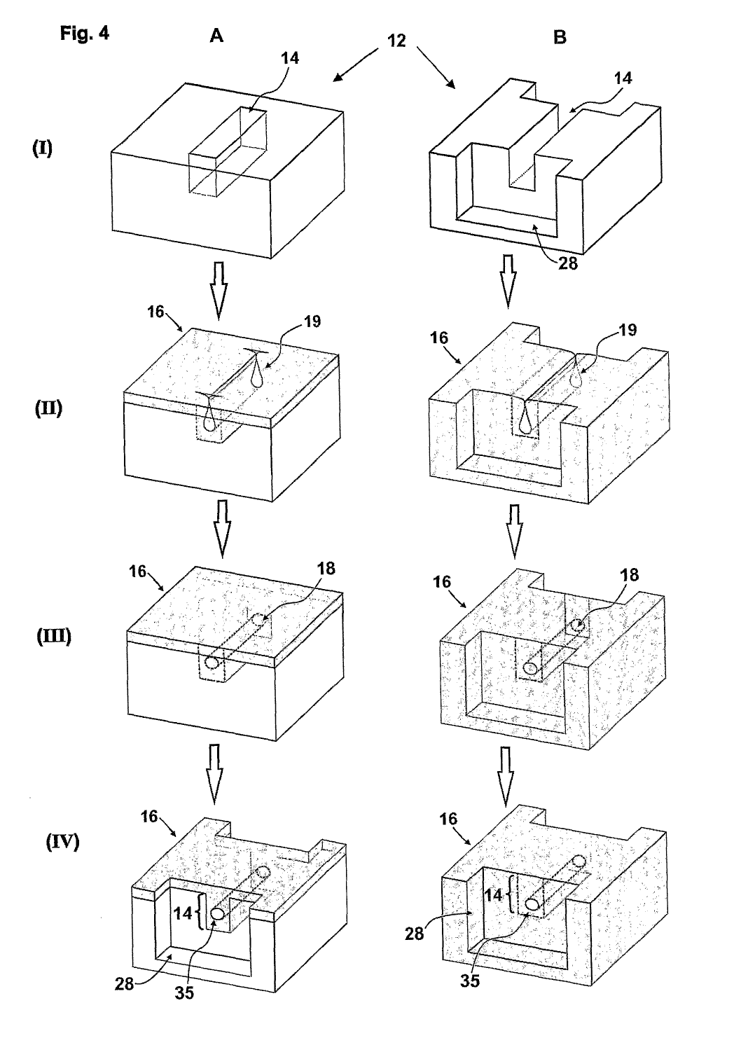

FIG. 3A shows a scanning electron microscopy (SEM) image depicting a perspective view of a completed device (60) according to the invention having two fluid chambers (62, 64) and a partitioning element (66) with a channel (68) buried therein. FIG. 3B to FIG. 3D show SEM close up views of the aperture of the channel (68), which is seen to define an area of at least substantially circular shape. While FIG. 3B and FIG. 3C show the aperture of the channel (68) of a device of the invention in a recess of a width of 2 μm, FIG. 3D shows the aperture of the channel (68) of a device of the invention in a recess of a width of 3 μm. The aperture geometry mainly depends on the aspect ratio of the recess (width/depth), the thickness of the PSG layer, and the annealing conditions. For the process parameters given below for FIG. 4A, 2 and 3 μm wide recesses produced microchannel diameters of 1.2 and 0.2 μm, respectively. Noteworthy, the microchannel diameter was generally observed to be inversely varying with the width of the recess. The lengths of the microchannels were lithographically set at 10, 20, and 50 μm. Each device was manually aligned and bonded to a replica-moulded PDMS layer which also contained reservoirs and fluidic interconnects.

FIG. 4 depicts a schematic overview of two embodiments of the method of the present invention of forming a microfluidic device (partitioning element). In method A (left hand side) a cuboid base substrate (12) is provided. In method B (right hand side) a cuboid base substrate (12) with two lateral recesses (28) is provided. A recess (14) is formed on the upper wall of the base substrate (I). In method B the recess (14) is open ended in that it stretches up to the two lateral recesses (28). Filling material is deposited on the top surface of the base substrate (A) or deposited or coated on the entire base substrate (B), thereby forming the filler member (16) with a void (19) in the recess (II). Deforming or re-flowing the filling material leads to a gradual change of the shape of the profile of the void in its cross section until its form approaches an at least substantially circular or an at least substantially elliptical shape. Thereby a channel (18) is formed that has a circumferential wall with a cross section of at least substantially circular or at least substantially elliptical-shaped profile (III). In method A two lateral recesses (28) are formed in the device by way of dry etching. Thereby the recess (14) becomes open-ended in that it stretches up to the two lateral recesses (28). At the same time apertures (35) of the channel are formed (IV). In method A at least a respective portion of the filling member occupying each of the two lateral recesses (28) are removed, such that apertures (35) of the channel are formed (IV). Briefly, in an exemplary method of the general scheme of FIG. 4A, on p-type silicon a recess was formed using deep ultraviolet (DUV) lithography followed by reactive ion etching (RIE) of silicon [(I)]. Thereby a 2 μm wide and 3.5 μm deep recess was defined. The recess was partially filled (to realize void in the trench) with phospho-silicate glass (PSG) containing ˜8% phosphorus in a plasma enhanced chemical vapour deposition (PECVD) system [(II)]. It is understood that other percentages than 8% phosphorus may be used where desired. The thickness of the phosphosilicate glass was set to be 4 μm, albeit other thicknesses may equally be used. Due to nonconformal step coverage, the PSG layer pinched off at the trench entrance before completely filling the trench, leaving a void trapped inside. The void was forced into a cylindrical shape by heat treatment called reflow of the PSG at 1150° C. for 30 min [(III)]. The reflow temperature may be adjusted as desired. Reducing the annealing temperature and the duration produced elliptic cross-section profiles while increasing them slightly decreased the cylindrical diameter. Excess PSG was removed from the substrate by a chemical mechanical polishing. A second lithography and an etching step were applied to create the fluid chambers and cut open both ends on each side of the buried microchannel [(IV)]. About 20 micrometer deep chambers were created in the silicon base substrate by first etching SiO₂ (4 micrometer; as deposited in step II above) followed by silicon etching. Optionally a wet-etching of PSG in buffered oxide etch (BOE) (1:6 ratio) for ˜1 min at room temperature may be carried out in order to increase the opened channel size. A final annealing in oxygen at 1150° C. for 15 min was carried out on some occasions, which helped to round off the aperture edge and insulate the exposed silicon by growing a 350 nm thick thermal SiO₂.

Briefly, in an exemplary method of the general scheme of FIG. 4B, a two-step lithographic patterning of silicon was carried out using deep ultraviolet (DUV) lithography followed by reactive ion etching (RIE) of silicon [(I)]. Thereby a 3.5 μm deep rectangular first recess was created. After a second lithography step, second, lateral recesses in the form of about 20-25 μm deep reservoirs were created on both sides of the trench-shaped first recess using DUV lithography followed by reactive ion etching of silicon. Subsequently, an about 4-6 μm thick layer of phosphosilicate glass (PSG), containing ˜8% phosphorus, was deposited on the substrate in a plasma enhanced chemical vapour deposition (PECVD) system. Thereby a trapped void was formed inside the trench [(II)]. Such void formation occurs due to non-conformal step coverage of the PSG layer which pinches off at the trench entrance before the trench is completely filled. The coated device was subjected to a Buffered Oxide Etchant [BOE (1:6)] wet-etching for 1 min at room temperature to open a small aperture before annealing. The time needed depends on the required aperture size. The void was purposely forced into a cylindrical shape by reflowing the PSG layer in an annealing step [(III)]. Typically reflow was done at 1150° C. for 30 minutes, but reflow temperature and time can be varied as desired. Optionally the reflowed device can be subjected to a further Buffered Oxide Etchant [BOE (1:6)] wet-etching for 1 min at room temperature depending on the required size of the hole.

The two embodiments of the method depicted in FIG. 4A and FIG. 4B may be combined by providing a base substrate with one lateral recesses. A recess may be formed on the upper wall of the base substrate, which spans one side of the upper wall and stretches up to the lateral recesses. Upon deposition of filling material on the base substrate the filler member with a void is formed. The channel formed by deforming or re-flowing the filling material reaches up to the lateral recess on one side of the base substrate. By removing a respective portion of the filling member occupying this lateral recesses a first apertures of the channel is formed. On the other side a lateral recess is formed in the microfluidic device by way of dry etching. Thereby a second apertures of the channel is formed. The obtained microfluidic device may include a channel with a tapered portion contiguous to the first aperture. The tapered portion may be conical along its length by a decrease in the size of its circumferential wall in terms of its width. Such a device may for example be used for positioning and immobilising a biological entity within the channel, such as at or in the tapered portion thereof.

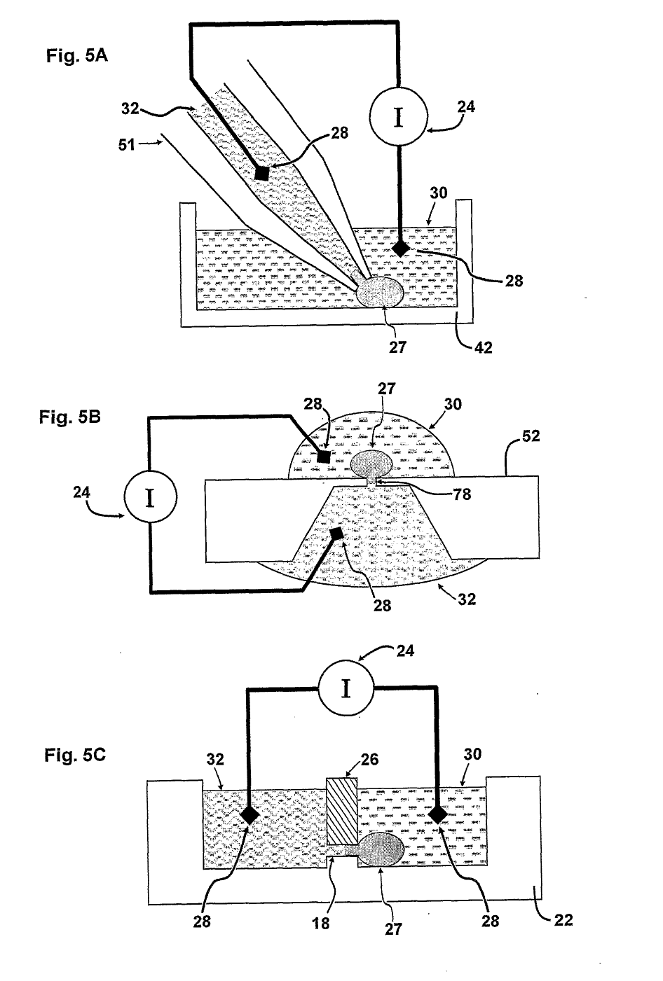

FIG. 5 depicts schematics of a patch clamp recording setup. FIG. 5A shows a classical setup using a glass micropipette (51) and a Petri dish (42). A cell is provided (27) in a sample solution (30). The cell, which is adhered to the bottom of a base substrate (22) is patched. The cell separates the sample solution (30) in the base substrate (22) from the sample solution in the interior (32) of the pipette. Sensing electrodes (28) are connected to a patch clamp amplifier (24) for carrying out electrical measurements. FIG. 5B shows a schematic of a planar patch setup. The cell (27) in a sample solution (30) is placed to the surface of a chip (52) such that it covers an aperture (78). Thereby it separates the sample solution (30) from the sample solution in a cavity (32) below the aperture (78). This approach avoids the requirement of a microscope, micromanipulator, and skilled operator by self-guiding and trapping a suspended cell (27) at a planar patch aperture on the base substrate (22). FIG. 5C shows a simplified diagram of a lateral cross sectional view of a lateral patch clamp setup, in which a partitioning element (26) arranged in a base substrate (22) between a first fluid chamber (30) which includes a sample solution containing a cell (27), and a second fluid chamber (32) which includes an electrolyte mixed with drained sample solution from the first fluid chamber (30), both fluid chambers being monolithically defined in the base substrate. The cell (27) is immobilised at the aperture of a channel (18) present in the lower portion of the partitioning element (26) through suction (suction device not shown). Sensing electrodes (28) positioned in the fluid chambers are connected to a patch clamp amplifier (24) for making electrical measurements, such as ion currents moving through the cell (27), or voltage potential across the cell (27). The patch clamp setup may further include a capping layer (not shown), which may serve in sealing the fluid chambers (30, 32) to assist suction applied on the cell.

FIG. 6 depicts SEM close up images of apertures of a channel obtained using the method shown in FIG. 4B. Both apertures define an area of at least substantially circular shape. White dashes indicate that the channel of the respective microfluidic device of FIG. 6A, including the portion contiguous to the aperture is of a cylindrical shape. In contrast thereto the channel of the respective microfluidic device of FIG. 6B has a tapered portion contiguous to the aperture. In this end portion the size of the circumferential wall in terms of its width, e.g. the diameter or the maximal diameter, decreases toward the aperture. The white dashes indicate the respective conical circumferential wall. This portion of the channel accordingly resembles the interior of a pulled conventional patch clamp pipette with a narrow mouth and widening body. Such conical channel configuration reduces the “access resistance” as compared to a cylindrical channel which is important in low-noise recording.

FIG. 7 shows a comparison of a tip of a pulled conventional micropipette for patch clamping (FIG. 7A) and two cut-out views of a microfluidic device according to the present invention (FIG. 7B & FIG. 7C). Both microfluidic devices depicted in FIG. 7B and FIG. 7C have a channel (18) that includes a conical portion (9) contiguous to the aperture (35), the width of which gradually decreases toward the apertures (35). The respective portions (9) of the channels thus resemble a pulled conventional patch clamp pipette. The aperture (35) of the channel depicted in FIG. 7B is included in a lateral wall portion (21) of the respective microfluidic device that is defined by the lateral wall of the filler member. This lateral wall portion (21) is at least essentially flat. In the device shown in FIG. 7C the aperture (35) of the channel is included in a lateral wall portion (21) thereof that is three-dimensionally structured. The surface of the lateral wall portion (21) contiguous to the aperture (35) is dished in that it defines a protrusion (33) surrounding the aperture (35). The channel aperture (35) is thereby located in a plane that differs from the plane defined by the lateral wall portion (21).

FIG. 8 depicts SEM images of a microfluidic device produced according to the method depicted in FIG. 4B before and after a focussed ion beam (FIB) cut along the line A-A′. FIG. 8A is a perspective close-up view of a portion of a microfluidic device including the portion of the filler member that includes the channel, which is located below the line A-A′. FIG. 8B is a perspective view showing the length of the cut circumferential wall of the channel. The first and the second aperture of the channel are each marked by a circle. As can be seen, the width of the end portions of the channel gradually decreases toward the apertures. FIG. 8A furthermore depicts two auxiliary channels (69) with apertures that are in vicinity to the aperture of the channel located below the line A-A′ (see below).

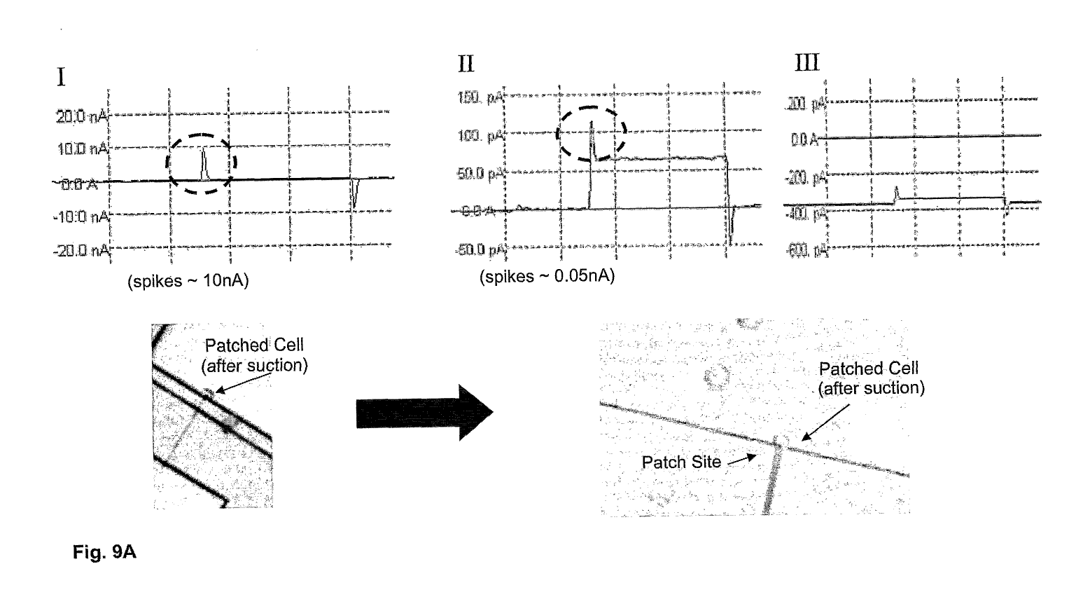

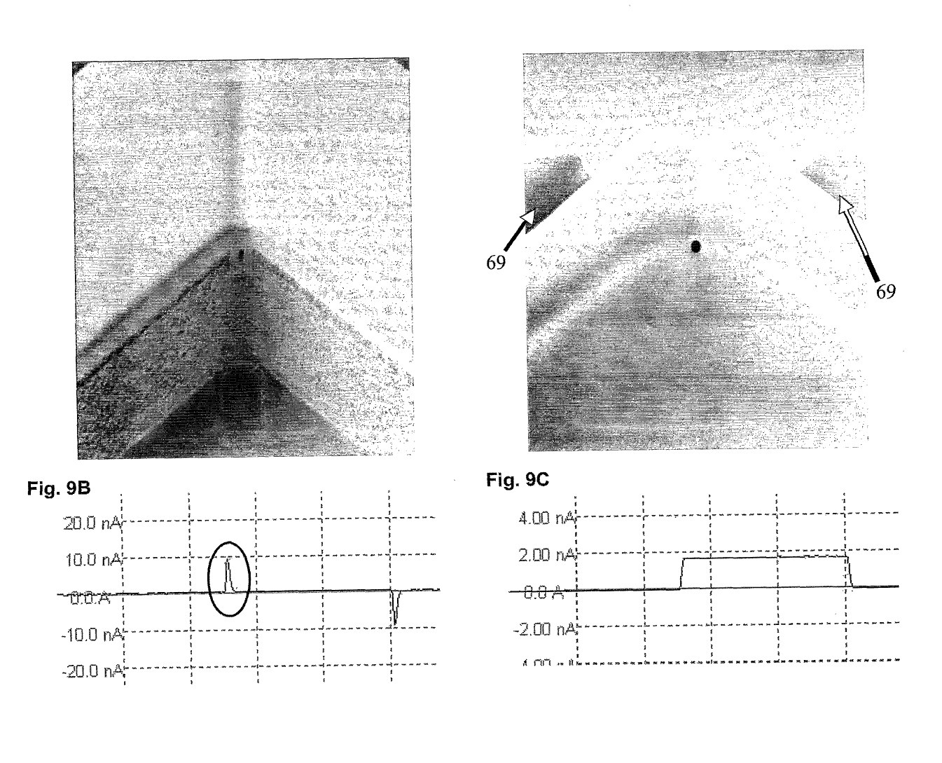

FIG. 9A depicts the use of a device as depicted in FIG. 2E (or FIG. 4) as a patch clamp chip. The device on the left (I) is made of silicon, whereas the device on the right (II, III) is made of glass. The devices included chambers as shown in FIG. 2E. Standard protocols were used for patch clamp measurements. As an example for the measurement depicted in (I) of FIG. 9A, chambers were primed with an electrolyte of a conductivity (σ) of 1.8 μm and in the following compositions (in mM): 150 NaCl, 2.8 KCl, 10 CaCl₂, 1 MgCl₂, 10 HEPES, and 2 mg/ml glucose, pH 7.2 (310 mOsm). Other solutions have successfully been used in the first and in the second fluid chamber (corresponding to the “bath solution” and the “pipette solution” of standard patch clamp methodology) in the same way as in conventional patch-clamp measurements. Electrical resistance (Rₒₚₑₙ) was measured across the two chambers via Ag/AgCl electrodes connected to a patch-clamp amplifier (EPC10, HEKA). For the microfluidic device obtained as shown in FIG. 4A, measurements agreed well with Rₒₚₑₙ=L/σπr² based on the microchannel geometry (L is length and r is radius). For the microfluidic device obtained as shown in FIG. 4B this can so far be confirmed on estimate-basis. Seal formation capability of the devices was tested on rat PC12 cells cultured according to a known protocol (Hahn, S. J., et al., Eur. J. Pharmacol. (1999) 367, 113). The cells were incubated with a fluorescent dye of 5 μg/ml calcein-AM (Invitrogen) at 37° C. for 15 min. They were then trypsinised, spun down (1000 rpm at 4° C. for 5 min), and resuspended in the electrolyte before being introduced into the bath chamber. When a cell was found within 50 μm reach of the patch aperture, it was repositioned and trapped by applying ˜25 kPa suction to the recording chamber through a manual syringe. It is noted that the suction pressure may vary depending on the fabrication method (including materials used) of the microfluidic device. In this regard less suction pressure is required when using a microfluidic device obtained as shown in FIG. 4B. In particular, in such embodiments bigger auxiliary channels can be used, thereby reducing the required suction pressure. A negative voltage bias was in the present example maintained to encourage seal formation. Such negative bias voltage may or may not be used. The current traces did not involve any compensation of the capacitive spikes which usually arise from charge coupling between the electrolyte and the silicon. It is understood that such spikes may be negligible or at least not as obvious when using a microfluidic device obtained as depicted in FIG. 4B, as can be taken from FIG. 9C. Measurements using CHO and RBL-1 cells were carried out with a similar protocol. The fluorescent dye was not always used but could have been included as described above. It is understood that the spun down parameter varies from cell type and cell culture conditions.

It may be desired to use glass, as a reduced capacitive coupling is observed and it may be easier to image cells under transmittance versus reflection mode. FIGS. 9B and 9C depict representative SEM images of a microfluidic device and typical electrical recordings from the device fabricated based on a process as depicted in FIG. 4 A (FIG. 9B) and depicted in FIG. 4B (FIG. 9C) (without capacitive compensation). The devices were formed from a base substrate of silicon, or (FIG. 9B) silicon with a thin SiO₂ insulation (˜0.35 um), grown during the last step depicted in FIG. 4A. Notably the current magnitudes (under the same voltage stimuli) differ significantly due to capacitive spikes. Such large capacitive spikes arise from charge coupling between the electrolyte and the silicon substrate. Removing the capacitive spikes electronically failed due to difficulty in estimating the distributed capacitance. Although depositing an extra thick dielectric layer avoids the capacitive coupling, it clogs up the patch aperture. On the other hand, attempts to thermally grow an oxide layer fail since high temperatures cause glass reflow and thereby the patch aperture to deform. The method depicted in FIG. 4B allows deposition of a thick layer of dielectric while preserving the desired appearance of the patch aperture. Hence, the capacitive spikes are minimised to an extent where they can be removed electronically (FIG. 9C). FIG. 9C furthermore depicts two auxiliary channels (69) (see also FIG. 8A and below).

FIG. 10A shows a summary of the electrical data of devices obtained by the method depicted in FIG. 4A that were tested as patch clamp chips. FIG. 10B depicts a summary of respective electrical data of devices obtained by the method depicted in FIG. 4B. 9 tests were performed using rat basophilic leukemia (RBL-1) cells. Noteworthy a giga seal could be achieved in 4 measurements. The two exemplary pictures show the device and setup before cell capture (I) and after cell capture (II).

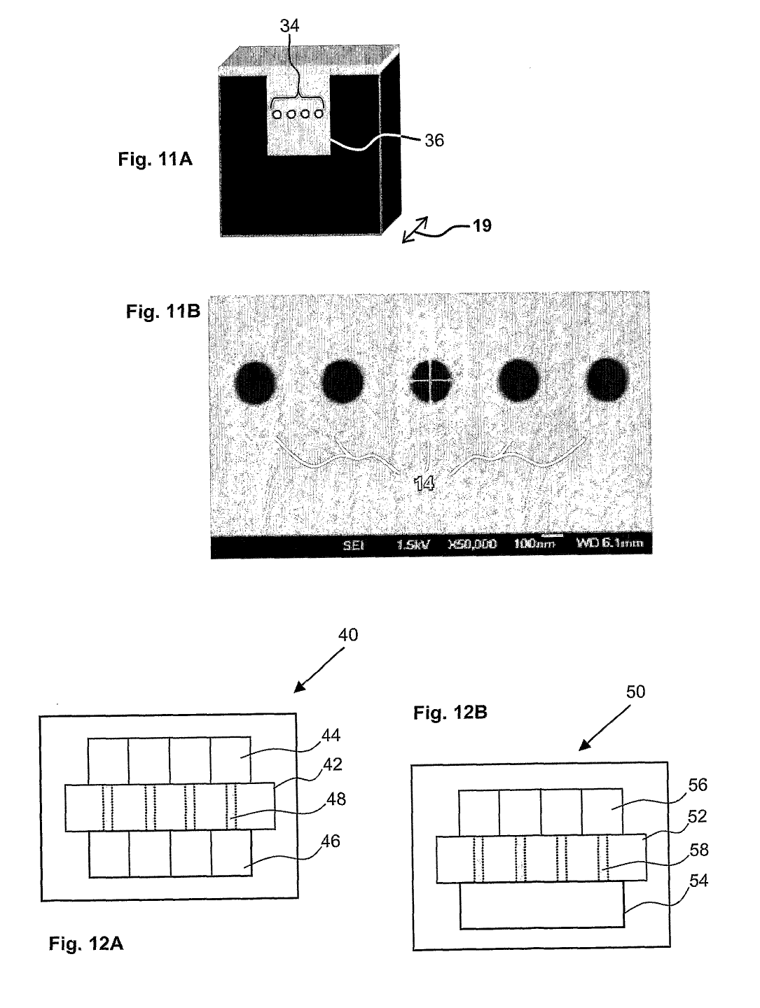

FIG. 11A shows a perspective view of an embodiment of a microfluidic device (serving as a partitioning element in a device as depicted e.g. in FIG. 2) of the invention, which includes a plurality of channels (34) in a filler member (36) that occupies a single recess. The arrow symbol (19) on the lower right of FIG. 11A indicates the lateral direction with respect to the device. FIG. 11B shows an electron microscope photograph of an actual partitioning element, in which a filler member occupies a plurality of recesses (14). One channel is arranged in each recess. The plurality of channels can be used to process a plurality of samples in parallel if so desired.

In a further embodiment as shown in FIG. 12A, a partitioning element (42) is arranged in a device (40) in between an array of first fluid chambers (44) and a respective array of second fluid chambers (46). Each first fluid chamber is separated from an adjacent first fluid chamber in the same array. Each channel (48) fluidly connects each first fluid chamber to its respective second fluid chamber. In this configuration, a large quantity of drugs, for example, can be individually screened for efficacy simultaneously. For this purpose, individual sets of sensing electrodes may be present to determine experimental measurements in each set of first and second fluid chambers. Alternatively, in a device (50), a single (common) first fluid chamber (54) may be present in the device (50) for receiving a sample (see FIG. 12B), which may contain a single type of cell. A partitioning element (52) with multiple channels (58) and having the same structure as that shown in FIG. 12A may be used. The first fluid chamber (54) is fluidly connected to an array of individually separate second fluid chambers (56). In this configuration, only one common ground electrode needs to be located in the first fluid chamber and as many independent sensing electrodes as the number of the second fluid chambers are disposed in each isolated second fluid chambers.

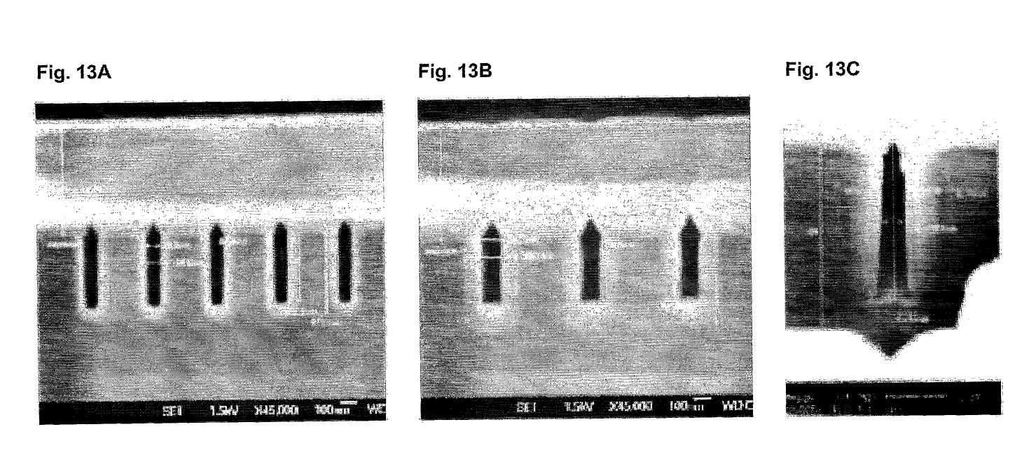

Recess sizes of less than 0.2 μm to 3 μm wide and <0.5 to 7 μm deep were fabricated according to the process described above. It is to be pointed out that recesses with smaller or larger dimensions than that obtained in the above experiments may be required to achieve different channel dimensions. Plasma Enhanced Chemical Vapour Deposition (PECVD) was used to fill doped silicon dioxide (PSG), at low pressure (2.5T) in the trenches (FIG. 13A). The wafers were then subjected to heat treatment at 1100° C. to 1200° C. for different timings depending on the final cross-section of channel required (FIG. 13B). Wafer surface is then planarized by Chemical Mechanical Planarization (CMP) or etching the excess PSG on the wafer surface followed by reservoir masking and etching (FIG. 13C). Such channels can also be used as the starting wafer to fabricate other device components like electrodes, interconnects and reservoirs, for example. The present process can also fabricate multiple vertically self-aligned channels. For example, after the construction of first channel, the top oxide may be removed partially and a second channel is fabricated over it.

FIG. 14 shows embodiments of immobilising a whole cell (53) using a device according to the present invention with a channel (18) that has a portion (9) contiguous to the aperture (35) that is conical along its length in that the size of the circumferential wall in terms of its width decreases toward the aperture (35). The aperture (35) of the channels (18) is included in a lateral wall portion (21). The two devices (FIG. 14A/FIG. 14B vs. FIG. 14C/FIG. 14D) correspond to the devices depicted in cut-out view in FIG. 7B and FIG. 7C. The cell (53) may be immobilised by way of suction and/or pressure force (similarly to conventional patch-clamping), indicated by arrows. In FIG. 14A and FIG. 14C the cell (53) is included in a fluid chamber (not shown), which is in both figures located on the right hand side of the lateral wall portion (21) and includes wall portion (21). The cell is immobilised on the aperture, thereby restricting the free movement of ions from the fluid chamber into the channel and vice versa (and thus into and from any other chamber, reservoir, channel, or other space that is in fluid connection with the channel). It also restricts free movement of ions from the second fluid chamber into the first fluid chamber. In FIG. 14B and FIG. 14D the cell (53) is included in the channel (18). Again the cell restricts the free movement of ions between the channel (and thus any other chamber, reservoir, channel, or other space that is in fluid connection with the channel) and a fluid chamber that may be located on the right hand side of the lateral wall portion (21) (and include wall portion (21)). It may in some embodiments be desired to immobilise the cell (53) inside the channel as depicted in FIG. 14B and FIG. 14D for ease of positioning the cell (53) before immobilising it, and also for higher stability of the cell's position after immobilisation.

FIG. 15 shows a close up image of a device according to the invention that includes auxiliary side channels (69) in addition to the channel with a circumferential wall (68) (used as a patch aperture in the present example). The auxiliary side channels are built around channel with a circumferential wall and include separate apertures. These channels can be arranged to be in fluid communication with individual devices such as containers, pumps etc. This allows the use of low pressure to apply suction. Thereby individual cells can be brought closer to the aperture of the channel with a circumferential wall and positioned there. At the same time cells can be selected by channeling unwanted cells into these channels and/or away from the aperture of the channel with a circumferential wall.

FIG. 16 depicts successive frames showing capturing an individual cell (marked by an arrow) at the aperture of the channel with a circumferential wall via applying controlled suction through the auxiliary side channels. Controlled suction applied through the auxiliary side channels attracts a nearby cell to the patch aperture. In frame T5 the cell is positioned at the respective aperture. Without the auxiliary channels, such suction applied through the channel with a circumferential wall itself might not generate an effective flow stream to attract such cell. In the status depicted in FIG. 4 as status I (A or B) the auxiliary channels may for example already be provided. Accordingly, typically the width of such side channels is designed sufficiently large so that during the fabrication of the device deposition of matter such as the filling material can not fill them up. Furthermore, the final cross-section of these side channels is generally larger than the cross-section of the channel with a circumferential wall (used as a patch aperture in the present example) for the applied suction to be effective. It is noted that a distance of 50 μm represents an example rather than an upper limit. In particular where a microfluidic device is used that has been obtained as shown in FIG. 4B, cells can be attracted that are further away than 50 μm.

Mathematical modelling of micro/nano-channel cross section dimension is carried out as follows. Let the non-conformal silicon oxide is filled in the trenches at temperature Tᵢ and pressure Pᵢ. This leads to a void in the trench with cross sectional area Aᵢ. Since the void created in the recess (trench) is at sub-atmospheric pressure, the void has tendency to reduce if the silicon oxide is softened. Depending on the softening conditions, the final dimension (A_{f}) of the void can be predicted. If the softening is done at temperature T_{f} and pressure P_{f}, from gas law:

(Pᵢ.Vᵢ)/Tᵢ=(P_{f}.V_{f})/T_{f} (1)

where, Vᵢ and V_{f} are the initial and final volume of the void.

But since the length of the void (trench) will remain unchanged, Vᵢ and V_{f} can be replaced by Aᵢ and A_{f} respectively in (1) to arrive at

(Pᵢ.Aᵢ)/Tᵢ=(P_{f}.A_{f})/T_{f} (2)

or, A_{f}=(Pᵢ/P_{f}).(T_{f}/Tᵢ).Aᵢ (3)

Say, in a typical case, BPSG is deposited at 400° C. and 50 Torr pressure. It is observed that it creates a void of about 6 μm² (6.0 μm×1.0 μm) cross sectional area, in the 2 μm wide and about 7.7 μm deep recess. This void can be deformed to circular cross sections after exposure to heating under reduced pressure or atmospheric pressure. Various examples of the channels obtained through this method is summarised in Table 2.

TABLE 2

| | Initial cross- | Reflow | Final cross- | Radius of | Actual radius |
|---|---|---|---|---|---|
| | sectional area | temperature | sectional area | channel, | of channel |
| S. No. | (Aᵢ), in μm² | (° C.) | (A_{f}), in μm² | (in μm) | (in μm) |
| 1 | 6.0 | 900 | 0.688 | 0.467 | 0.406 |
| 2 | 6.0 | 950 | 0.717 | 0.477 | 0.443 |
| 3 | 6.0 | 1000 | 0.746 | 0.487 | 0.505 |

In summary, the present invention is capable of producing lateral channels with circular or elliptical cross-section, which provides the minimum surface/frictional resistance and better electrical sealing. The invention is also capable of forming channels with cross-sectional diameter in the range of microns to nanometer while current methods are only good for either producing micro-channels or nano-channels. The channel cross section dimensions can be predicted and controlled precisely by varying fabrication conditions. The fabrication processes are fully CMOS compatible and can therefore be implemented at existing silicon foundries. Channel fabrication cost is low as no specialized tools/processes like electron beam lithography, wafer bonding, laser source, polymers, etc. are used.

The invention can also be used to fabricate multiple, self-aligned channels, both laterally and vertically.

The listing or discussion of a previously published document in this specification should not necessarily be taken as an acknowledgement that the document is part of the state of the art or is common general knowledge. All documents listed are hereby incorporated herein by reference in their entirety. The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognised that various modifications are possible within the scope of the invention claimed. Additional objects, advantages, and features of this invention will become apparent to those skilled in the art upon examination of the foregoing examples and the appended claims. Thus, it should be understood that although the present invention is specifically disclosed by exemplary embodiments and optional features, modification and variation of the inventions embodied therein herein disclosed may be resorted to by those skilled in the art, and that such modifications and variations are considered to be within the scope of this invention. In addition, where features or aspects of the invention are described in terms of Markush groups, those skilled in the art will recognise that the invention is also thereby described in terms of any individual member or subgroup of members of the Markush group.

1. A microfluidic device comprising:
a base substrate having a recess defined therein by at least two opposing lateral walls and a base wall,
a filler member having at least a portion thereof occupying the recess, and
a channel defined in the portion of the filler member occupying the recess,
wherein the channel comprises a first aperture and a second aperture, the first aperture being arranged on a first lateral wall of the filler member, and the second aperture being arranged on a second lateral wall of the filler member, said first lateral wall of the filler member being arranged in opposing relationship with the second lateral wall of the filler member, and at least a portion of the first and the second lateral walls of the filler member being at least substantially perpendicular to the opposing lateral walls defining the recess, and
wherein the channel is defined by a circumferential wall, wherein the cross-section of said circumferential wall in at least a portion of the channel has an at least substantially circular or an at least substantially elliptical-shaped profile.

2. The microfluidic device of claim 1, wherein at least one of the first aperture and the second aperture of the channel defines an area of at least substantially circular or elliptical shape.

3. The microfluidic device of claim 1, wherein the circumferential wall of the channel has a portion contiguous to the first aperture, said portion being conical along its length in that the size of the circumferential wall in terms of its width decreases toward the first aperture.

4.-8. (canceled)

9. The microfluidic device of claim 1, wherein the channel is arranged laterally within the filler member.

10. The microfluidic device of claim 1, wherein the longitudinal axis of the channel is at least substantially perpendicular to the first lateral wall and/or the second lateral wall of the filler member.

11. The microfluidic device of claim 1, wherein the first lateral wall of the filler member defines a wall portion of the base substrate.

12. The microfluidic device of claim 11, wherein the wall portion of the base substrate defined by the lateral wall of the filler member is comprised in a lateral wall of the base substrate.

13. The microfluidic device of claim 11, wherein the wall portion of the base substrate defined by the lateral wall of the filler member is comprised in a lateral recess of the base substrate.

14. The microfluidic device of claim 11, wherein the wall portion of the base substrate defined by the lateral wall of the filler member comprises a lateral recess.

15. The microfluidic device of claim 14, wherein the lateral recess comprises the first aperture of said channel.

16. The microfluidic device of claim 13, wherein the lateral recess is arranged at least essentially perpendicular to the recess of which at least a portion is occupied by the filler member.

17.-18. (canceled)

19. The microfluidic device of claim 13, wherein the lateral recess comprises a circumferential wall and an inlet, thereby defining a fluid chamber.

20. The microfluidic device of claim 13, comprising a first and a second lateral recess on two at least essentially opposing sides of the base substrate, wherein the first lateral recess comprises at least a portion of the first lateral wall of the filler member and the second lateral recess comprises at least a portion of the second lateral wall of the filler member,
wherein the first lateral recess defines a first fluid chamber and the second lateral recess defines a second fluid chamber, the first fluid chamber being in fluid communication with the second fluid chamber via the channel.

21. The microfluidic device of claim 20, wherein the first fluid chamber and the second fluid chamber are monolithically defined in the base substrate.

22. The microfluidic device of claim 20, further comprising a sensing electrode disposed in the first fluid chamber and a reference electrode disposed in the second fluid chamber.

23. The microfluidic device of claim 22, further comprising electrophysiological measurement circuitry in communication with the sensing and reference electrodes.

24. The microfluidic device of claim 1, wherein the filler member defines the entire surface of the base substrate.

25.-28. (canceled)

29. The microfluidic device of claim 1, wherein the base substrate is of a material that is less deformable than the material of the filler member.

30. The microfluidic device of claim 29, wherein said material of the base substrate is less deformable than the material of the filler member under conditions of elevated temperature and/or reduced pressure.

31. The microfluidic device of claim 1, wherein the filler member comprises a dielectric material.

32. (canceled)

33. The microfluidic device of claim 1, wherein the microfluidic device further comprises an auxiliary channel, wherein the auxiliary channel comprises a first aperture and a second aperture, wherein the first aperture is arranged on the same side of the microfluidic device as the first aperture of the channel that is defined in the portion of the filler member occupying the recess.

34. The microfluidic device of claim 33, wherein the first aperture of the auxiliary channel is arranged in a lateral wall portion of the base substrate, wherein said lateral wall portion of the base substrate comprises the first aperture of the channel that is defined in the portion of the filler member occupying the recess.

35. The microfluidic device of claim 33, wherein the first aperture of the auxiliary channel is arranged in a recess of the lateral wall portion of the base substrate, wherein said recess comprises the first aperture of the channel that is defined in the portion of the filler member occupying the recess.

36. The microfluidic device of claim 33, wherein the first aperture of the auxiliary channel is juxtaposed to the first aperture of the channel that is defined in the portion of the filler member occupying the recess.

37. The microfluidic device of claim 1, wherein the portion of the filler member occupying the recess of the base substrate comprises a plurality of channels, each of said plurality of channels comprising a first aperture comprised in a first lateral wall portion of the filler member and a second aperture comprised in a second lateral wall portion of the filler member.

38. The microfluidic device of claim 1 further comprising:
a plurality of recesses defined in the substrate,
the filler member having corresponding portions thereof arranged to occupy at least a portion of each recess, and
a plurality of channels, arranged such that a channel is comprised in each of said corresponding portions of the filler member, wherein each of said plurality of channels comprises a first aperture comprised in a first lateral wall portion of the filler member and a second aperture comprised in a second lateral wall portion of the filler member.

39. The microfluidic device of claim 38, wherein all first lateral wall portions of the filler member that comprise the aperture of a respective channel defined in the plurality of recesses are aligned so as to define a common plane.

40. The microfluidic device of claim 39, wherein the plurality of first lateral wall portions of the filler member defines a wall portion of the base substrate, said wall portion of the base substrate being comprised in a lateral recess of the base substrate.

41. The microfluidic device of claim 39, wherein the plurality of first lateral wall portions of the filler member defines a plurality of wall portions of the base substrate, each wall portion of said plurality of wall portions being comprised in a lateral recess of the base substrate, thereby defining a plurality of lateral recesses of the base substrate.

42. A microfluidic device comprising:
a base substrate comprising a recess, wherein the recess comprises two opposing lateral walls and a base wall,
a filler member, wherein a portion of the filler member is comprised in the recess of the base substrate, and
a channel defined in the portion of the filler member comprised in the recess,
wherein the channel comprises a first aperture and a second aperture, the first aperture being arranged on a first lateral wall of the filler member, and the second aperture being arranged on a second lateral wall of the filler member, the first lateral wall of the filler member being arranged in opposing relationship with the second lateral wall of the filler member,
wherein the channel is defined by a circumferential wall, wherein the cross-section of the circumferential wall in at least a portion of the channel has an at least substantially circular or an at least substantially elliptical-shaped profile, the circumferential wall having a portion contiguous to the first aperture, said portion being conical along its length in that the size of the circumferential wall in terms of its width decreases toward the first aperture.

43. The microfluidic device of claim 42, wherein at least a portion of the first and the second lateral walls of the filler member are at least substantially perpendicular to the opposing lateral walls defining the recess.

44-53. (canceled)

54. A microfluidic device comprising:
a first fluid chamber for containing a particle to be tested, a second fluid chamber that is fluidly separated from the first fluid chamber by means of a partitioning element, said partitioning element comprising:
a base substrate having a recess defined therein,
a filler member having a portion thereof occupying the recess, and
a channel defined in the portion of the filler member occupying the recess,
wherein the channel comprises a first aperture and a second aperture, the first aperture being arranged on a first lateral wall of the filler member, and the second aperture being arranged on a second lateral wall of the filler member, said first lateral wall of the filler member being arranged in opposing relationship with the second lateral wall of the filler member, and at least a portion of said first lateral wall and said second lateral wall of the filler member being at least substantially perpendicular to the opposing lateral walls defining the recess, and
wherein the channel is defined by a circumferential wall, wherein the cross-section of the circumferential wall in at least a portion of the channel has an at least substantially circular or an at least substantially elliptical-shaped profile.

55.-66. (canceled)

67. A microfluidic device comprising:
a first fluid chamber for containing a particle to be tested, a second fluid chamber that is fluidly separated from the first fluid chamber by means of a partitioning element, said partitioning element comprising:
a base substrate having a recess defined therein,
a filler member having a portion thereof occupying the recess, and
a channel defined in the portion of the filler member occupying the recess,
wherein the channel comprises a first aperture and a second aperture, the first aperture being arranged on a first lateral wall of the filler member, and the second aperture being arranged on a second lateral wall of the filler member, said first lateral wall of the filler member being arranged in opposing relationship with the second lateral wall of the filler member,
wherein the channel is defined by a circumferential wall, wherein the cross-section of the circumferential wall in at least a portion of the channel has an at least substantially circular or an at least substantially elliptical-shaped profile, the circumferential wall having a portion contiguous to the first aperture, said portion being conical along its length in that the size of the circumferential wall in terms of its width decreases toward the first aperture.

68.-71. (canceled)

72. A method of forming a microfluidic device, comprising:
providing a base substrate,
forming a recess on a surface of the base substrate,
filling said recess with a filling material, and
subjecting the filling material comprised in the recess to a condition that causes it to deform, thereby forming a channel in the portion of the filling material occupying the recess.

73. The method of claim 72, wherein forming the recess comprises etching a surface of the base substrate.

74. The method of claim 72, wherein the recess is open-ended in that it stretches up to at least one side of the base substrate.

75. The method of claim 72, wherein filling the recess is achieved by covering the base substrate with the filling material.

76. The method of claim 72, wherein filling of the recess with filling material is carried out via a deposition process.

77.-78. (canceled)

79. The method of claim 72, wherein the filling material comprises doped silicate glass.

80. The method of claim 72, further comprising forming a void within the portion of filling material that is occupying the recess, such that the void is arranged to be extended along the recess.

81. The method of claim 72, wherein filling the recess with a filling material comprises depositing the filling material into the recess in a manner that causes the filling material to pinch together at the opening end of the recess, thereby forming a void within the portion of the filling material occupying the recess.

82. The method of claim 80, wherein the recess is open-ended in that it stretches up to at least one side of the base substrate, such that said void has an end adjacent to the open-ended side of the recess,
wherein subjecting the filling material to a condition that causes it to deform comprises allowing a channel to be formed from the void, wherein the channel has a circumferential wall with a portion adjacent to the open-ended side of the recess, said portion being conical along its length in that the size of the circumferential wall in terms of its width decreases toward the open-ended side of the recess.

83. The method of claim 72, wherein the condition to which the substrate is subjected comprises heating under reduced pressure.

84-85. (canceled)

86. A method of forming a microfluidic device, comprising:
providing a base substrate,
forming a first recess on a surface of the base substrate,
forming a second recess on a surface of the base substrate, wherein said surface differs from the surface on which the first recess is formed,
filling the first recess with the filling material, and
subjecting the filling material comprised in the recess to a condition that causes it to deform, thereby forming a channel in the portion of the filling material occupying the first recess.

87.-109. (canceled)

110. A method of forming a microfluidic device, comprising:
providing a base substrate,
forming a recess on a surface of the base substrate, such that said recess is formed to be open-ended in that it stretches up into at least one side of the base substrate,
covering the base substrate with a filling material, and
subjecting the filling material comprised in the recess to a condition that causes it to deform, thereby forming a channel in the portion of the filling material occupying the recess.

111.-147. (canceled)
